SEQUENCE LISTING (25 sequences)

SEQ ID NO: 1 - protein, 714 aa, S. enterica
MSRIIMLIPTGTSVGLTSVSLGVIRAMERKGVRLSVFKPIAQPRAGGDAPDQTTTIVRANSTLPAAEPLKMSHVESLLSSNQKDVLMEEIIANYHANTKDAEVVLVEGLVPTRKHQFAQSLNYEIAKTLNAEIVFVMSQGTDTPEQLNERIELTRSSFGGAKNTNITGVIINKLNAPVDEQGRTRPDLSEIFDDSSKAQVIKIDPAKLQESSPLPVLGAVPWSFDLIATRAIDMARHLNATIINEGDIKTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYVNAEWIESLTATSERSRRLSPPAFRYQLTELARKAGKRVVLPEGDEPRTVKAAAICAERGIATCVLLGNPDEINRVAASQGVELGAGIEIVDPEVVRESYVARLVELRKSKGMTEPVAREQLEDNVVLGTLMLEQDEVDGLVSGAVHTTANTIRPPLQLIKTAPGSSLVSSVFFMLLPEQVYVYGDCAINPDPTAEQLAEIAIQSADSAIAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQASQQQQ

SEQ ID NO: 2 - protein, 714 aa, E. coli 0157H7
MSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIDSLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 3 - protein, 714 aa, E. coli K12
MSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 4 - protein, 717 aa, Y. pestis
MSRTIMLIPTGTSVGLTSVSLGVIRSMEQKGVSLSVFKPIAQPRAGNDAPDQTTTIIRANSSITAAEPLNMNYVETLLSSNQQDVLMEEIVARYHENTKDAEVVLVEGLVPTRKHQFANALNYEIAKTLNAEIVFVIALGNDSPDQLKERIELARSSFGGSKNKNITGVIINKLNAPVDEQGRTRPDLSEIFDDSTKASVANIDPSQLFANSPIPVLGCVPWSFELIATRAIDMAKHLNARIINEGDIKTRRVKSVTFCARSIPHMLEHFRPGSLLVTSADRPDVLVSACLAAMNGVEIGAILLTGGYAIDDRINNLCERAFQTGLPVFMVDTNTWQTSLSLQSFNLEVPADDHERVEKLQNYVASHISSEWIDSLTAASERPRRLSPPAFRYELTELARKAGKRIVLPEGDEPRTIKAASICAERGIATCVLLGNPEEIQRVATSQGVELGKGVEIIDPVAVREQYVPRLVELRKSKGMTEVVAREQLEDNVVLGTLMLEKGEVDGLVSGAVHTTANTIRPPLQLIKTAPGSSLVSSVFFMLLPDQVLVYGDCAINPDPTAEQLSEIAIQSADSAAAFGIEPRVAMISYSTGNSGAGSDVEKVREATRLAQEKRPDLIIDGPLQYDAAIMADVAKSKAPNSPVAGRATVFIFPDLNTGNTTYKAVQRSADLISIGPMLQGMRKPVNDLSRGALVDDIVYTVALTAIQSAQADSAAS

SEQ ID NO: 5 - protein, 500 aa, N. meningitidis
MAKVLIVPVSAGLDASAVAQAFAKALDAQIFQAVDATAETLLAQGKSDDWFDALVGKVAALDAANLVIEGIAPDADKIYLAGKNVELALSLDAAAVFAVRSDNADADELANRLNLAKQFFAAAPGVLEGFVVDGAAASVAEAAAEKTGLTFFGSSDALKDVSVLAGREAKRLSPAQFRYNLIDFARQADKRIVLPEGAEPRTVQAAAICHEKGIARCVLLAKREEVEAVAKERGISLPDSLEIIDPASLVEQYVEPMCELRKSKGLTPEDARKQLQDTVVLGTMMMAQNDVDGLVSGAVHTTANTIRPALQLIKTAPGASLVSSVFFMLLPNQVLVFGDCAVNPNPTAQQLADIAIQSADSAKAFGIDPKVAMISYSTVNSGSGPDVDTVIEATKLAQEKRPDLAIDGPLQYDAATVPGVGKSKAPGSPVAGQATVLVFPDLNTGNCTYKAVQRSANVLSVGPLLQGLRKPVNDLSRGALVEDIVFTIALTAVQAKQMEG

SEQ ID NO: 6 - protein, 690 aa, M. tuberculosis
MADSSAIYLAAPESQTGKSTIALGLLHRLTAMVAKVGVFRPITRLSAERDYILELLLAHTSAGLPYERCVGVTYQQLHADRDDAIAEIVDSYHAMADECDAVVVVGSDYTDVTSPTELSVNGRIAVNLGAPVLLTVRAKDRTPDQVASVVEVCLAELDTQRAHTAAVVANRCELSAIPAVTDALRRFTPPSYVVPEEPLLSAPTVAELTQAVNGAVVSGDVALREREVMGVLAAGMTADHVLERLTDGMAVITPGDRSDVVLAVASAHAAEGFPSLSCIVLNGGFQLHPAIAALVSGLRLRLPVIATALGTYDTASAAASARGLVTATSQRKIDTALELMDRHVDVAGLLAQLTIPIPTVTTPQMFTYRLLQQARSDLMRIVLPEGDDDRILKSAGRLLQRGIVDLTILGDEAKVRLRAAELGVDLDGATVIEPCASELHDQFADQYAQLRKAKGITVEHAREIMNDATYFGTMLVHNCHADGMVSGAAHTTAHTVRPALEIIKTVPGISTVSSIFLMCLPDRVLAYGDCAIIPNPTVEQLADIAICSARTAAQFGIEPRVAMLSYSTGDSGKGADVDKVRAATELVRAREPQLPVEGPIQYDAAVEPSVAATKLRDSPVAGRATVLIFPDLNTGNNTYKAVQRSAGAIAIGPVLQGLRKPVNDLSRGALVDDIVNTVAITAIQAQGVHE

SEQ ID NO: 7 - protein, 704 aa, P. aeruginosa
MHTFFIAPTGFGVGLTSISLGLLRALERAGLKVGFFKPIAQLHPGDLGPERSSELVARTHGLDTPKPLPLAQVERMLGDGQLDELLEEIISLYQRAAADKDVVIVEGMVPTRHASYAARVNFHLAKSLDAEVILVSAPENETLTELTDRIEIQAQLFGGPRDPKVLGVILNKVRGEADAANAEDGVADFARRLTEHSPLLRDDFRLIGCIPWQDELNAARTRDIADLLSARVINAGDYEQRRVQKIVLCARAVPNTVQLLKPGVLVVTPGDRDDIILAASLAAMNGVPLAGLLLCSDFPPDPRIMELCRGALQGGLPVLSVATGSYDTATNLNRMNKEIPVDDRERAERVTEFVAGHIDFEWLKQRCGTPRELRLSPPAFRYQVVQRAQKAGKRIVLPEGSEPRTVQAAAICQARGIARCVLLAKPEEVQAVAQAQGIVLPEGLEIIDPDLVRQRYVEPMVELRKGKGLNAPMAEQQLEDSVVLATMMLALDEVDGLVSGAIHTTASTIRPALQLIKTAPGYNLVSSVFFMLLPDQVLVYGDCAVNPDPSASDLAEIAVQSAASAQAFGIPARVAMISYSTGDSGSGVDVDKVREATRLAREQRPDLLIDGPLQYDAAAIASVGRQKAPNSPVAGQATVFIFPDLNTGNTTYKAVQRSADCVSVGPMLQGLRKPVNDLSRGALVEDIVYTIALTAIQADAQAPA

SEQ ID NO: 8 - protein, 711 aa, H. influenzae
MSRTIILIPVSTGVGLTSISLGLIHSLEQKGTKVAFMKPVSQPSTGEDKLDRTTSIIRTSTSLETAEPFMLSVAESLIGQNQSDVLLEKIVANHQQLTKNNDIVVVEGLIPTRKHGYANSINYEIAQALDAEIVLVAAPATETPTELKDRVEAAASLFGGKNNPNLLGVVVNKFNAPVDESGRTRPDLAEIFDSFQHNHISETEVNKLFAGSAIKLLACVPWNANLIATRAIDLVKHLGASIINEGEINRRIRGITFCARSLPNMVEHFRAGSLLVASADRPDVLVAAALAASNGIEIGGILLTGGYKIDAQINKLCRPTFEKAKLPIFRIEGNTWQTALSLQSFNLEVPVDDKERIENIKQYISQHFNADFINNLVADSSRLPRLSPPAFRFQLTELARAAKKRIVLPEGDEPRTIKAAVLCAERGIAECVLLADPASVQRVAEAQGVKLGKGITIINPADVRENYVDRLVELRKAKGMTETAAREQLEDTVVLGTMMLEANEVDGLVSGAVHTTANTIRPPMQIIKTAPGSSIVSSIFFMLLPDQVLVYGDCAVNPDPTAEQLAEIAIQSADSAKAFGIDPKVAMISYSTGTSGSGADVEKVKEATRIAKEKRPDLLIDGPLQYDAAVMEDVARSKAPNSPVAGKATVFVFPDLNTGNTTYKAVQRSADLVSIGPMLQGMRKPVNDLSRGALVDDIVYTIALTAIQATQ

SEQ ID NO: 9 - protein, 519 aa, H. pylori
MQSLWIYPEDTEVLGAACKSLLKALKPRYQKIALFSPISGGCEGFGECESLSSLEVHSAIDKQKALELVSTAQEELLFETILKRYDELQSTHDFVINLGYAPKFFLNALLDLNTILAKHLNAPVVAVAQTSLDHLKAMHSHILKKEAPFAIGLFVGETLEKPHFLSASLCKQQCELEASAIENLLQTKSEIITPLAFQRSLEKKAKKQIKKVVLPESEDERILKAAHRLNLMGAVGLILLGDKEAINSQAKNLNLNLENVEIINPNTSHYREEFAKSLYELRKSKGLSEQEAERLALDKTYFATMLVHLGYAHAMVSGVNHTTAETIRPALQIIKTKPGVSLVSSVFLMCLDTQVFVFGDCAIIPNPSPKELAEIATTSAQTAKQFNIAPKVALLSYATGNSAQGEMIDKINEALTIVQKLDPQLEIDGPLQFDASIDKGVAKKKMPNSQVAGQASVFIFPDLNAGNIAYKAVQRSAKAVAIGPILQGLNKPINDLSRGALVEDIVNTVLISAIQAQDY

SEQ ID NO: 10 - protein, 335 aa, C. perfingens
MNMELMKQIWESAKNNRKKIVLPEGDEERTLVASQRIKEEGLADVYLVGSEQVIREKAEALGVNLEGVNIVDPETSDKLDTYINEFYELRKAKGMTVEKAGKIVRDPLYFGTMMVKMGDADGMVSGAVHTTGDLLRPGLQIIKTAPGVSVVSSFFIMMVPGSQYGEGGMLLFSDCAVNPNPNADQLAAIAIATADTAKNLCKMDPKVAMLSFSTMGSADHDLVTKVRVATEKAKELRPDLDIDGELQLDAAIVGKVASQKAPNSKVAGNANVLVFPDLQAGNIGYKLVQRFANAEAIGPVCQGFAKPINDLSRGCSSEDIVNVVAITAVQAQATK

SEQ ID NO: 11 - protein, 697 aa, Synechocystis
MTSSLYLSTTEARSGKSLVVLGILDLILKKTTRIAYFRPIIQDPVNGKHDNNIILVLENFRLQQTYTDSFGLYFHEAVSLASDGAIDQVLDRILAKYRHLADQVDFILCEGSDYLGEESAFEFDLNTTIAKMLNCPILLLGNAMGNTIADSLQPIDMALNSYDQESCQVVGVIINRVQPELATEIQAQLEQRYGDRPMVLGTIPQDIMLKSLRLREIVSGLNAQVLSGADLLDNLVYHHLVVAMHIAHALHWLHEKNTLIITPGDRGDIILGVMQAHRSLNYPSIAGILLTADYHPEPAIMKLIEGLPDAPPLLLTSTHTHETSARLETLHPALSPTDNYKIRHSIALFQQQIDGEKLLNYLKTIRSKGITPKLFLYNLVQAATAAQRHIVLPEGEEIRILKAAASLINHGIVRLTLLGNIEAIEQTVKINHIDLDLSKVRLINPKTSPDRERYAETYYQLRKHKGVTLAMARDILTDISYFGTMMVHLGEADGMVSGSVNTTQHTVRPALQIIKTQPGFSLVSSVFFMCLEDRVLVYGDCAVNPDPNAEQLAEIALTSAATAKNFGIEPRVALLSYSSGSSGQGADVEKVRQATAIAKEREPDLALEGPIQYDAAVDSTVAAQKMPGSAVAGKATVFIFPDLNTGNNTYKAVQRETKAIAIGPILQGLNKPVNDLSRGCLVEDIINTVVITALQVK

SEQ ID NO: 12 - protein, 333 aa, M. thermophila
MVTFLEKISERAKKLNKTIALPETEDIRTLQAAAKILERGIADIVLVGNEADIKALAGDLDLSKAKIVDPKTYEKKDEYINAFYELRKHKGITLENAAEIMSDYVYFAVMMAKLGEVDGVVSGAAHSSSDTLRPAVQIVKTAKGAALASAFFIISVPDCEYGSDGTFLFADSGMVEMPSVEDVANIAVISAKTFELLVQDVPKVAMLSYSTKGSAKSKLTEATIASTKLAQELAPDIAIDGELQVDAAIVPKVAASKAPGSPVAGKANVFIFPDLNCGNIAYKIAQRLAKAEAYGPITQGLAKPINDLSRGCSDEDIVGAVAITCVQAAAQDK

SEQ ID NO: 13 - protein, 338 aa, S. enterica
MIIERARELAVRAPARVVFPDALDERVLKAAHYLQQYGLARPVLVASPFALRQFALSHRMAMDGIQVIDPHSNLSMRQRFAQRWLARAGEKTPPDAVEKLSDPLMFAAAMVSAGEADVCIAGNLSSTANVLRAGLRVIGLQPGCKTLSSIFLMLPQYAGPALGFADCSVVPQPTAAQLADIALASADTWRAITGEEPRVAMLSFSSNGSARHPNVANVQQATELVRERAPQLLVDGELQFDAAFVPEVAAQKAPDSPLQGRANVMIFPSLEAGNIGYKITQRLGGYRAVGPLIQGLAAPLHDLSRGCSVQEIIELALVAAVPRQADVSRERSLHTLVE

SEQ ID NO: 14 - DNA, 29 bp, artificial
  synthetic oligonucleotide
tgtaacccgggcccaaaagactgtaacga

SEQ ID NO: 15 - DNA, 28 bp, artificial
  synthetic oligonucleotide
tcacctctagacctgacaaggcgttcac

SEQ ID NO: 16 - DNA, 7138 bp, artificial
  genetically engineered plasmid
gctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattcgagctcggtacccgggcccaaaagacggtaacgaaagaggataaaccgtgtcccgtattattatgctgatccctaccggaaccagcgtcggcctgaccagcgtcagcctcggcgtcatccgtgctatggaacgcaaaggcgttcgtctgagcgtctttaagcctatcgcccagcctcgcgctggcggcgatgcgcctgaccagaccaccactatcgttcgcgcgaactctaccctgccggcggctgaaccgctgaagatgagccacgttgaatctctgctctccagcaaccagaaagacgtgctgatggaagagatcatcgcgaactaccatgcgaataccaaagacgcggaagtggtgctggttgaaggtctggttccgacccgtaaacatcagttcgctcagtctctgaactatgaaatcgcgaaaacgctgaatgcggaaatcgtttttgtcatgtctcagggtaccgacacgccagaacagctgaacgagcgtatcgaactgacgcgcagcagcttcggcggcgcgaaaaacaccaacatcaccggtgttattatcaacaaactgaatgcgccggttgatgaacaaggccgtactcgcccggatctgtcggagatctttgacgactcttccaaagcgcaggtgatcaaaatcgatcctgctaaactgcaggaatccagcccgctgccggttctgggcgcggtgccgtggagcttcgacctgattgcgacccgcgctatcgatatggcgcgtcacctgaacgccaccatcattaacgaaggcgatatcaaaacccgccgcgttaaatccgtcactttctgcgcgcgtagtattccgcatatgctggaacatttccgcgcaggctcgttgttagtgacctccgctgaccgtccggacgtactggtcgcagcttgcctggccgcgatgaacggcgtagaaatcggcgccctgttgctgaccggcggctatgaaatggacgcgcgcatttctaaactgtgcgaacgcgcattcgccaccggtctgccggtctttatggtgaacaccaacacctggcagacttcgctgagcctgcaaagcttcaatctggaagtcccggttgatgaccatgagcgcatcgagaaagttcaggaatacgtcgcgaactacgttaacgctgagtggatcgagtcgctgaccgccacttccgagcgtagccgtcgtctctctccgccggcgttccgctaccaactgactgagctggcgcgtaaagccggtaaacgcgtagtgctgccggaaggcgacgaaccgcgtaccgtcaaagcggcggcaatctgcgctgaacgcggcatcgccacttgcgtactgctgggcaacccggatgaaatcaaccgcgtcgcggcatctcagggcgttgagctgggcgcaggtattgaaatcgtcgatccggaagtggtgcgtgaaagctatgtcgctcgcctggttgagctgcgtaagagcaaaggcatgaccgaaccggttgcccgcgaacagctggaagacaacgtggtgctcggcacgctgatgctggagcaagacgaagtcgacggcctggtttccggcgcggttcataccaccgccaacaccatccgtccgccgctgcagcttattaaaacggcgccgggtagctccctggtctcttctgtgttctttatgctactgccggaacaggtttacgtttacggcgactgcgcaatcaacccagacccgaccgcagagcagctggcagaaatcgcgattcagtctgcggattccgccattgccttcggcatcgaaccgcgtgtggcaatgctctcctactccaccggcacctctggcgcgggcagcgacgtagagaaagtacgtgaagcgacgcgtctggcgcaggaaaaacgtcctgacctgatgatcgacggtccgttgcagtacgacgccgcggtcatggctgacgtagcgaaatccaaagcgccgaactcgccggttgcgggtcgcgctaccgtgttcattttcccggatctgaacaccggtaacaccacctacaaagcggtacagcgttctgccgacctgatctccatcgggccgatgttgcagggtatgcgcaagccggtgaacgacctgtcccgtggcgcgctggttgacgatatcgtctacaccatcgccctgacggcgatccaggcttctcagcagcagcagtaacagtaaaagctaatgccggatggcggcgtgaacgccttgtccggtctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatg

SEQ ID NO: 17 - DNA, 32 bp, artificial
  synthetic oligonucleotide
gaggataaaccatggcccgtattattatgctg

SEQ ID NO: 18 - DNA, 28 bp, artificial
  synthetic oligonucleotide
tcacctctagacctgacaaggcgttcac

SEQ ID NO: 19 - DNA, 8122 bp, artificial
  genetically engineered plasmid
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatggttcaactagtggttctggtcatcaccatcaccatcactccgcgggtctggtgccacgcggtagtactgcaattggtatgaaagaaaccgctgctgctaaattcgaacgccagcacatggacagcccagatctgggtaccggtggtggctccggtattgagggacgcgggtccatggcccgtattattatgctgatccctaccggaaccagcgtcggcctgaccagcgtcagcctcggcgtcatccgtgctatggaacgcaaaggcgttcgtctgagcgtctttaagcctatcgcccagcctcgcgctggcggcgatgcgcctgaccagaccaccactatcgttcgcgcgaactctaccctgccggcggctgaaccgctgaagatgagccacgttgaatctctgctctccagcaaccagaaagacgtgctgatggaagagatcatcgcgaactaccatgcgaataccaaagacgcggaagtggtgctggttgaaggtctggttccgacccgtaaacatcagttcgctcagtctctgaactatgaaatcgcgaaaacgctgaatgcggaaatcgtttttgtcatgtctcagggtaccgacacgccagaacagctgaacgagcgtatcgaactgacgcgcagcagcttcggcggcgcgaaaaacaccaacatcaccggtgttattatcaacaaactgaatgcgccggttgatgaacaaggccgtactcgcccggatctgtcggagatctttgacgactcttccaaagcgcaggtgatcaaaatcgatcctgctaaactgcaggaatccagcccgctgccggttctgggcgcggtgccgtggagcttcgacctgattgcgacccgcgctatcgatatggcgcgtcacctgaacgccaccatcattaacgaaggcgatatcaaaacccgccgcgttaaatccgtcactttctgcgcgcgtagtattccgcatatgctggaacatttccgcgcaggctcgttgttagtgacctccgctgaccgtccggacgtactggtcgcagcttgcctggccgcgatgaacggcgtagaaatcggcgccctgttgctgaccggcggctatgaaatggacgcgcgcatttctaaactgtgcgaacgcgcattcgccaccggtctgccggtctttatggtgaacaccaacacctggcagacttcgctgagcctgcaaagcttcaatctggaagtcccggttgatgaccatgagcgcatcgagaaagttcaggaatacgtcgcgaactacgttaacgctgagtggatcgagtcgctgaccgccacttccgagcgtagccgtcgtctctctccgccggcgttccgctaccaactgactgagctggcgcgtaaagccggtaaacgcgtagtgctgccggaaggcgacgaaccgcgtaccgtcaaagcggcggcaatctgcgctgaacgcggcatcgccacttgcgtactgctgggcaacccggatgaaatcaaccgcgtcgcggcatctcagggcgttgagctgggcgcaggtattgaaatcgtcgatccggaagtggtgcgtgaaagctatgtcgctcgcctggttgagctgcgtaagagcaaaggcatgaccgaaccggttgcccgcgaacagctggaagacaacgtggtgctcggcacgctgatgctggagcaagacgaagtcgacggcctggtttccggcgcggttcataccaccgccaacaccatccgtccgccgctgcagcttattaaaacggcgccgggtagctccctggtctcttctgtgttctttatgctactgccggaacaggtttacgtttacggcgactgcgcaatcaacccagacccgaccgcagagcagctggcagaaatcgcgattcagtctgcggattccgccattgccttcggcatcgaaccgcgtgtggcaatgctctcctactccaccggcacctctggcgcgggcagcgacgtagagaaagtacgtgaagcgacgcgtctggcgcaggaaaaacgtcctgacctgatgatcgacggtccgttgcagtacgacgccgcggtcatggctgacgtagcgaaatccaaagcgccgaactcgccggttgcgggtcgcgctaccgtgttcattttcccggatctgaacaccggtaacaccacctacaaagcggtacagcgttctgccgacctgatctccatcgggccgatgttgcagggtatgcgcaagccggtgaacgacctgtcccgtggcgcgctggttgacgatatcgtctacaccatcgccctgacggcgatccaggcttctcagcagcagcagtaacagtaaaagctaatgccggatggcggcgtgaacgccttgtcaggtctagaggtgaaatcactagtgaattctgtacaggccttggcgcgcctgcaggcgagctccgtcgacaagcttgcggccgcactcgagcaccaccaccaccaccaccaccactaattgattaatacctaggctgctaaacaaagcccgaaaggaagctgagttggctgctgccaccgctgagcaataactagcataaccccttggggcctctaaacgggtcttgaggggttttttgctgaaaggaggaactatatccggat

SEQ ID NO: 20 - protein, 992 aa, artificial
  genetically engineered chimeric protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDGSTSGSGHHHHHHSAGLVPRGSTAIGMKETAAAKFERQHMDSPDLGTGGGSGIEGRGSMARIIMLIPTGTSVGLTSVSLGVIRAMERKGVRLSVFKPIAQPRAGGDAPDQTTTIVRANSTLPAAEPLKMSHVESLLSSNQKDVLMEEIIANYHANTKDAEVVLVEGLVPTRKHQFAQSLNYEIAKTLNAEIVFVMSQGTDTPEQLNERIELTRSSFGGAKNTNITGVIINKLNAPVDEQGRTRPDLSEIFDDSSKAQVIKIDPAKLQESSPLPVLGAVPWSFDLIATRAIDMARHLNATIINEGDIKTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYVNAEWIESLTATSERSRRLSPPAFRYQLTELARKAGKRVVLPEGDEPRTVKAAAICAERGIATCVLLGNPDEINRVAASQGVELGAGIEIVDPEVVRESYVARLVELRKSKGMTEPVAREQLEDNVVLGTLMLEQDEVDGLVSGAVHTTANTIRPPLQLIKTAPGSSLVSSVFFMLLPEQVYVYGDCAINPDPTAEQLAEIAIQSADSAIAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQASQQQQ

SEQ ID NO: 21 - DNA, 30 bp, artificial
  synthetic oligonucleotide
tcgcaactctctactatttctccatacccg

SEQ ID NO: 22 - DNA, 30 bp, artificial
  synthetic oligonucleotide
cgggtatggagaaatagtagagagttgcga

SEQ ID NO: 23 - DNA, 7138 bp, artificial
  genetically engineered plasmid
gctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactatttctccatacccgtttttttgggctagcgaattcgagctcggtacccgggcccaaaagacggtaacgaaagaggataaaccgtgtcccgtattattatgctgatccctaccggaaccagcgtcggcctgaccagcgtcagcctcggcgtcatccgtgctatggaacgcaaaggcgttcgtctgagcgtctttaagcctatcgcccagcctcgcgctggcggcgatgcgcctgaccagaccaccactatcgttcgcgcgaactctaccctgccggcggctgaaccgctgaagatgagccacgttgaatctctgctctccagcaaccagaaagacgtgctgatggaagagatcatcgcgaactaccatgcgaataccaaagacgcggaagtggtgctggttgaaggtctggttccgacccgtaaacatcagttcgctcagtctctgaactatgaaatcgcgaaaacgctgaatgcggaaatcgtttttgtcatgtctcagggtaccgacacgccagaacagctgaacgagcgtatcgaactgacgcgcagcagcttcggcggcgcgaaaaacaccaacatcaccggtgttattatcaacaaactgaatgcgccggttgatgaacaaggccgtactcgcccggatctgtcggagatctttgacgactcttccaaagcgcaggtgatcaaaatcgatcctgctaaactgcaggaatccagcccgctgccggttctgggcgcggtgccgtggagcttcgacctgattgcgacccgcgctatcgatatggcgcgtcacctgaacgccaccatcattaacgaaggcgatatcaaaacccgccgcgttaaatccgtcactttctgcgcgcgtagtattccgcatatgctggaacatttccgcgcaggctcgttgttagtgacctccgctgaccgtccggacgtactggtcgcagcttgcctggccgcgatgaacggcgtagaaatcggcgccctgttgctgaccggcggctatgaaatggacgcgcgcatttctaaactgtgcgaacgcgcattcgccaccggtctgccggtctttatggtgaacaccaacacctggcagacttcgctgagcctgcaaagcttcaatctggaagtcccggttgatgaccatgagcgcatcgagaaagttcaggaatacgtcgcgaactacgttaacgctgagtggatcgagtcgctgaccgccacttccgagcgtagccgtcgtctctctccgccggcgttccgctaccaactgactgagctggcgcgtaaagccggtaaacgcgtagtgctgccggaaggcgacgaaccgcgtaccgtcaaagcggcggcaatctgcgctgaacgcggcatcgccacttgcgtactgctgggcaacccggatgaaatcaaccgcgtcgcggcatctcagggcgttgagctgggcgcaggtattgaaatcgtcgatccggaagtggtgcgtgaaagctatgtcgctcgcctggttgagctgcgtaagagcaaaggcatgaccgaaccggttgcccgcgaacagctggaagacaacgtggtgctcggcacgctgatgctggagcaagacgaagtcgacggcctggtttccggcgcggttcataccaccgccaacaccatccgtccgccgctgcagcttattaaaacggcgccgggtagctccctggtctcttctgtgttctttatgctactgccggaacaggtttacgtttacggcgactgcgcaatcaacccagacccgaccgcagagcagctggcagaaatcgcgattcagtctgcggattccgccattgccttcggcatcgaaccgcgtgtggcaatgctctcctactccaccggcacctctggcgcgggcagcgacgtagagaaagtacgtgaagcgacgcgtctggcgcaggaaaaacgtcctgacctgatgatcgacggtccgttgcagtacgacgccgcggtcatggctgacgtagcgaaatccaaagcgccgaactcgccggttgcgggtcgcgctaccgtgttcattttcccggatctgaacaccggtaacaccacctacaaagcggtacagcgttctgccgacctgatctccatcgggccgatgttgcagggtatgcgcaagccggtgaacgacctgtcccgtggcgcgctggttgacgatatcgtctacaccatcgccctgacggcgatccaggcttctcagcagcagcagtaacagtaaaagctaatgccggatggcggcgtgaacgccttgtccggtctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatg

SEQ ID NO: 24 - DNA, 18 bp, Artificial
  synthetic oligonucleotide
gtcgcccgaattcaacaa

SEQ ID NO: 25 - DNA, 18 bp, Artificial
  synthetic oligonucleotide
ttaaagggtaccagaacg

(19) United States
(12) Patent Application Publication
(10) Pub. No.: US 2006/0275320 A1
(43) Pub. Date: Dec. 7, 2006

(54) Method of attenuating bacterial virulence by targeting the phosphotransacetylase N-terminal domain

(75) Inventors: Jorge C. Escalante-Semerena, Madison, WI (US); Shaun R. Brinsmade, Madison, WI (US)

Correspondence Address: QUARLES & BRADY LLP, 411 E. WISCONSIN AVENUE, SUITE 2040, MILWAUKEE, WI, 53202-4497, US

(73) Assignee: Wisconsin Alumni Research Foundation

(21) Appl. No.: 11/502,985

(22) Filed: Aug. 11, 2006

Related U.S. Application Data
(62) Division of application No. 10/944,020, filed on Sep. 17, 2004.
(60) Provisional application No. 60/504,497, filed on Sep. 18, 2003.

(51) Int. Cl. A61K 39/02; G01N 33/554; C12Q 1/18; C12N 1/21

(52) U.S. Cl. 424/200.1; 435/252.3; 435/7.32; 435/32

(57) ABSTRACT

It is disclosed that the N-terminal domain of a long form bacterial phosphotransacetylase is important for bacterial virulence. Various isolated polypeptides, antibodies, isolated nucleic acids, vectors, and host cells that relate to the N-terminal domain of a long form bacterial phosphotransacetylase are disclosed. Further disclosed are methods of using the N-terminal domain of a long form bacterial phosphotransacetylase to screen for agents that can attenuate bacterial virulence and methods for attenuating bacterial virulence by targeting the N-terminal domain of a long form bacterial phosphotransacetylase.

### CROSS-REFERENCE TO RELATED APPLICATIONS

This application is a divisional application of U.S. patent application Ser. No. 10/944,020, filed on Sep. 17, 2004, which claims the benefit of U.S. provisional application Ser. No. 60/504,497, filed on Sep. 18, 2003.

### STATEMENT REGARDING FEDERALLY SPONSORED RESEARCH OR DEVELOPMENT

This invention was made with United States government support awarded by the following agency: NIH Grant No. GM40313. The United States government has certain rights in this invention.

### BACKGROUND OF THE INVENTION

Acetate is used as a source of carbon and energy by prokaryotes occupying diverse habitats such as soil, where acetate is one of the most abundant fatty acids, or the gastrointestinal tract of humans, where the concentration of acetate can reach high levels. Acetate needs to be activated into acetyl-CoA, which feeds directly into the TCA cycle, to enter central metabolism and to generate energy.

In enteric bacteria such as Escherichia coli and Salmonella enterica, acetate is activated into acetyl-CoA via either one of two pathways. The first pathway requires the involvement of acetate kinase (AckA, EC 2.7.2.1) and phosphotransacetylase (Pta, EC 2.3.1.8). AckA catalyzes the conversion between acetate and acetyl-phosphate and Pta catalyzes the conversion between acetyl-phosphate and acetyl-CoA. Both enzymes can catalyze the conversions in either direction. When acetate is present in high concentrations in the environment (≧30 mM acetate), AckA and Pta drive the reactions towards the synthesis of acetyl-CoA. This pathway is considered to be the low-affinity pathway for acetate activation. The second pathway for acetate activation requires the activity of the ATP-dependent acetate:CoA ligase (AMP forming; EC 6.2.1.1; aka acetyl-CoA synthetase) encoded by the acs gene. Acs is required when the concentration of acetate in the environment is low (≦10 mM acetate), thus this pathway is considered to be the high-affinity pathway for acetate activation.

It is well known in the art that some bacteria contain a long form of Pta and some contain a short form. The long form has an N-terminal domain and a C-terminal domain. The short from is homologous to the C-terminal domain of the long form. Both forms are catalytically active.

### BRIEF SUMMARY OF THE INVENTION

The present invention is based on the inventors' discovery that the N-terminal domain of a long form bacterial phosphotransacetylase (Pta) is important for bacterial virulence. In one aspect, the present invention relates to an isolated polypeptide that comprises an N-terminal domain of a long form bacterial Pta with the proviso that a polypeptide comprising a full length Pta is excluded.

In another aspect, the present invention relates to an antibody that binds to a long form bacterial Pta at its N-terminal domain.

In another aspect, the present invention relates to an isolated nucleic acid comprising a nucleotide sequence that encodes an N-terminal domain of a long form bacterial Pta with the proviso that a nucleic acid comprising a nucleotide sequence that encodes a full length Pta is excluded.

In another aspect, the present invention relates to a method of using the N-terminal domain of a long form bacterial Pta to screen for agents that can attenuate bacterial virulence.

In another aspect, the present invention relates to a method for attenuating bacterial virulence by targeting the N-terminal domain of a long form bacterial Pta.

### BRIEF DESCRIPTION OF THE SEVERAL VIEWS OF THE DRAWINGS

FIG. 1 shows alignment of Pta (including EutD) proteins from various bacterial species and strains. The amino acid sequences for S. enterica LT2, E. coli 0157H7, E. coli K12, Y. pestis KIM, N. meningitidis Z2491, M. tuberculosis, P. aeruginosa PAO1, H. influenzae, H. pylori, C. perfingens Strain 13, Synechocystis PCC, M. thermophila, S. enterica LT2 EutD are provided as SEQ ID NO:1-13, respectively, in the sequence listing.

FIG. 2 shows that EutD can restore growth of a pta/acs mutant on acetate. pta acs/pBAD30: Salmonella enterica strain harboring lesions in both acs and pta and transformed with control pBAD30; pta⁺ acs⁺/pBAD30: Salmonella enterica strain with intact acs and pta and transformed with control pBAD30; pta acs/pT7-7: Salmonella enterica strain harboring lesions in both acs and pta and transformed with control pT7-7 (Tabor, S. 1990. Expression using the T7 RNA polymerase/promoter system., p. 16.2.1.-16.2.11. In F. M. Ausubel, R. Brent, R. E. Kingston, D. D. Moore, J. G. Seidman, J. A. Smith, and K. Struhl (ed.), Current Protocols in Molecular Biology, vol. 2. Wiley Interscience, New York.); pta acs/pMthpta⁺: Salmonella enterica strain harboring lesions in both acs and pta and transformed with pMthpta⁺, a pT7-7 based plasmid containing the M. thermophila pta gene; pta acs/pSeeutD⁺: Salmonella enterica strain harboring lesions in both acs and pta and transformed with pSeeutD⁺.

### DETAILED DESCRIPTION OF THE INVENTION

It is disclosed here that for a bacterium that contains a long form phosphotransacetylase (Pta), the N-terminal domain of the Pta is important for virulence. This conclusion is based on the observation that knocking out a long form Pta from bacteria reduced both bacterial virulence and their ability to rely on acetate as a nutrient and that introducing a short form Pta into the Pta knock-out bacteria restored their ability to use acetate but not their virulence. The identification of the N-terminal domain of a long Pta as being involved in bacterial virulence provides new drug targets and drug-screening tools.

The N-terminal domain of a long Pta is homologous to CbiP, an enzyme involved in the B12 synthesis pathway. In particular, CbiP is an amino transferase involved in the late steps of de novo corrin ring biosynthesis. However, the Pta N-terminal domain lacks a key catalytic motif of CbiP. Thus, the Pta N-terminal domain is believed to be enzymatically inactive in the B12 synthesis but possesses the ability to bind to compounds similar to B12. B12 belongs to a family of compounds called cyclic tetrapyrroles, which includes heme. Heme contains one iron atom, the scavenging and uptake of which plays a role in bacterial virulence. Bacterial cells need heme to respire to oxygen. Without intending to be limited by theory, the inventors propose that the N-terminal domain of a long Pta can bind heme and thus serves as a heme sensor for directing the conversion between acetyl-phosphate and acetyl-CoA in one direction or the other to promote bacterial proliferation.

In one aspect, the present invention relates to an isolated polypeptide comprising an N-terminal domain of a long form bacterial Pta with the proviso that a polypeptide comprising a full length Pta is excluded.

Pta (Pta, EC 2.3.1.8) is a well known bacterial enzyme that enables bacteria to use acetate as an energy source. Almost all species of bacteria have this enzyme and the amino acid sequences are available in the GenBank of NCBI. For example, the Pta amino acid sequences for P. syringae, P. aeruginosa PAO1, N. meningitidis Z2491, S. enterica LT2, E. coli 0157H7, E. coli K12, Y. pestis KIM, H. influenzae, H. pylori, Synechocystis PCC, M. tuberculosis, C. perfingens Strain 13, M thermophila, S. enterica LT2 EUTD can be found with GenBank Accession Numbers AAO54696, AAG04224, NP_{—}283633, NP_{—}461280, BAB36604, AAC75357, AAM85189, P45107, Q9ZKU4, NP_{—}441027, P96254, NP_{—}562641, 1QZTD, and NP_{—}461401, respectively. The Pta's of different species have been identified by homology to other Pta sequences and the enzymatic activity can be confirmed with routine knock-out studies. One easy way to obtain Pta amino acid sequences from species other than those provided above is to conduct a BLAST search in the GenBank of NCBI using any of the above Pta sequences (Altschul, Stephen F., Thomas L. Madden, Alejandro A. Schaffer, Jinghui Zhang, Zheng Zhang, Webb Miller, and David J. Lipman. 1997. Gapped BLAST and PSI-BLAST: a new generation of protein database search programs, Nucleic Acids Res. 25:3389-3402). For example, one can use BLASTP 2.2.9 (Protein:Protein BLAST, available at NCBI website), Matrix BLOSUM45, and Gap Penalties of 10 (Existence) and 3 (Extension) to conduct the blast search.

It is also well known in the art that bacterial Pta's can be categorized into the long form family and the short form family. A short Pta is typically shorter than 350 or 400 amino acids and a long Pta is typically longer than 400 or 450 amino acids. A long form Pta has an N-terminal domain and a C-terminal catalytic domain. A short form Pta is homologous to the C-terminal domain of a long form Pta. Both forms are catalytically active. Examples of long form Pta include but are not limited to those from P. syringae, P. aeruginosa PAO1, N. meningitidis Z2491, S. enterica LT2, E. coli 01571H7, E. coli K12, Y. pestis KIM, H. influenzae, H. pylori, Synechocystis PCC, and M. tuberculosis. Examples of short form Pta include but are not limited to those from C. perfingens Strain 13 and M. thermophila. An alignment of some of the above long and short Pta's is provided in FIG. 1.

The N-terminal domain of the S. enterica LT2 Pta is defined as a fragment that starts at amino acid one and ends at an amino acid between amino acid 359 and amino acid 381. The N-terminal domains of Pta's from other species also start at amino acid one and end at an amino acid position corresponding to that between amino acid 359 and 381 of the S. enterica LT2 Pta when they are aligned with the S. enterica LT2 Pta using the MegAlign (Ver. 5.06) multiple alignment program bundled with the DNAStar software (DNASTAR, Inc., Madison, Wis.). A ClustalW analysis was performed using a Gonnet 250 protein weight matrix with gap penalty=10, and gap length penalty=0.20. An N-terminal domain in the shortest form is from amino acid one to amino acid 360 for the S. enterica LT2 Pta and from amino acid one to an amino acid position corresponding to amino acid 360 of the S. enterica LT2 Pta for other Pta's when they are aligned as described above. For example, the N-terminal domain in the shortest form for those from P. aeruginosa PAO1, N. meningitidis Z2491, S. enterica LT2, E. coli 0157H7, E. coli K 12, Y. pestis KIM, H. influenzae, H. pylori, Synechocystis PCC, and M. tuberculosis are amino acids 1 to 340, 1 to 167, 1 to 350, 1 to 351, 1 to 351, 1 to 350, 1 to 350, 1 to 188, 1 to 335, and 1 to 306, respectively.

The term “polypeptide” and the term “protein” are used interchangeably in the specification and claims.

The term “isolated polypeptide” or “isolated nucleic acid” used in the specification and claims means a polypeptide or nucleic acid isolated from its natural environment or prepared using synthetic methods such as those known to one of ordinary skill in the art. Complete purification is not required in either case. Amino acid or nucleotide sequences that flank a polypeptide or nucleic acid in nature can but need not be absent from the isolated form. A polypeptide and nucleic acid of the invention can be isolated and purified from normally associated material in conventional ways such that in the purified preparation the polypeptide or nucleic acid is the predominant species in the preparation. At the very least, the degree of purification is such that the extraneous material in the preparation does not interfere with use of the polypeptide or nucleic acid of the invention in the manner disclosed herein. The polypeptide or nucleic acid is preferably at least about 85% pure, more preferably at least about 95% pure and most preferably at least about 99% pure.

In addition, an isolated polypeptide in the present invention refers to a peptide molecule that neither the whole molecule or a part thereof is identical to any naturally occurring protein.

Further, an isolated nucleic acid in the present invention refers to a nucleic acid that neither the whole molecule nor a part thereof is identical to any naturally occurring nucleic acid or to any fragment of a naturally occurring genomic nucleic acid spanning one or more genes. The term therefore covers, for example, (a) a DNA that has the sequence of part of a naturally occurring genomic DNA molecule but which is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library. An isolated nucleic acid molecule can be modified or unmodified DNA or RNA, whether fully or partially single-stranded or double-stranded or even triple-stranded. A modified nucleic acid molecule can be chemically or enzymatically induced and can include so-called non-standard bases such as inosine.

In another aspect, the present invention relates to an antibody that binds to a long form bacterial Pta at its N-terminal domain. The antibody can be a monoclonal or polyclonal antibody. The monoclonal antibody can be a murine antibody, a chimeric antibody, or a humanized antibody. It is well within the capability of a skilled artisan to generate the above antibodies.

In another aspect, the present invention relates to an isolated nucleic acid comprising a nucleotide sequence that encodes an N-terminal domain of a long form bacterial Pta with the proviso that a nucleic acid comprising a nucleotide sequence that encodes a full length Pta is excluded. Optionally, the nucleic acid can further comprise a transcription control sequence (e.g., a promoter) operably linked to the nucleotide sequence that encodes the N-terminal domain. Both native and non-native transcription control sequences can be employed. A host cell comprising the above nucleic acids is also within the scope of the present invention. In a preferred embodiment, the host cell is a bacterial cell.

In another aspect, the present invention relates to a method for screening for agents that can attenuate bacterial virulence. The method involves providing a polypeptide that contains the N-terminal domain of a long form Pta, exposing the polypeptide to a test agent, and determining whether the agent binds to the N-terminal domain. If an agent binds to the N-terminal domain, it is very likely that the agent can attenuate virulence of a bacterium that contains the corresponding or a related Pta. It is possible that not 100% of the positively identified agents by this screening assay will be able to attenuate bacterial virulence and hence further virulence assays may be necessary to confirm the activity. The screening assay will, however, significantly reduce the number of more expensive virulence assays before a positive agent can be identified. A skilled artisan is familiar with the suitable virulence assays that can be used. One example is provided in the examples below. As discussed above, since eukaryotic cells do not have Pta, an anti-bacterial agent identified by this screen should have minimal side effects on a human or non-human animal.

Any polypeptide that contains the N-terminal domain of a Pta can be used in the screening assay. For example, the N-terminal domain can be flanked by native or non-native sequences. The flanking sequences can but do not have to assist in purification, stabilization and detection of the polypeptide. The preferred polypeptides include the N-terminal domain of various long Pta's and the long Pta's themselves.

There are many systems in the art that a skilled artisan is familiar with for assaying the binding between a polypeptide and an agent. Any of these systems can be used in the method of the present invention. Detailed experimental conditions can be readily determined by a skilled artisan. For example, a polypeptide that contains a Pta N-terminal domain can be provided on a suitable substrate and exposed to a test agent. The binding of the agent to the polypeptide can be detected either by the loss of ability of the polypeptide to bind to an antibody or by the labeling of the polypeptide if the agent is labeled with radioactivity, fluorescence or other features. In another example, a polypeptide that contains a Pta N-terminal domain can be expressed in a host cell, e.g., a bacterial cell, and the cell is then exposed to a test agent. Next, the polypeptide can be isolated, e.g., by immunoprecipitation or electrophoresis, and the binding between the polypeptide and the agent can be determined. As mentioned above, one way to determine the binding between the polypeptide and the agent is to label the agent with radioactivity or fluorescence so that the polypeptide that binds to the agent is radioactive or fluorescent. Detailed experimental conditions can be readily determined by a skilled artisan. It should be noted that when a Pta N-terminal domain used in the screening assay has flanking sequences, it may be necessary to confirm that an agent binds to the N-terminal domain rather than the flanking sequences, which can be readily accomplished by a skilled artisan.

In another aspect, the present invention relates to a method for attenuating the virulence of a bacterium that contains a long form Pta. The method involves exposing the bacterium to a molecule that can bind to the N-terminal domain of the Pta at a dose sufficient to attenuate virulence. Since eukaryotic cells do not contain Pta, this Pta-based method should have minimal side effects on human and non-human animals. In one embodiment of the method, the N-terminal domain-binding molecule is an antibody to the domain. Since the amino acid sequences of the long form Pta in various bacterial strains are known, a skilled artisan can readily generate monoclonal or polyclonal antibodies to the N-terminal domain.

In another embodiment, a molecule obtained from the screening assays described above is used to bind to the N-terminal domain to attenuate virulence.

The invention will be more fully understood upon consideration of the following non-limiting examples.

### EXAMPLE 1

### Plasmids for Functional Studies

This is an example on how to construct a plasmid that can be employed to transform bacteria for conducting Pta-related functional assays such as that described in example 5 below. Although the example only illustrates the cloning of the wild-type S. enterica pta, it is understood that other genetic sequences of interest such as mutated pta's and wild-type pta's from other species can be cloned similarly.

Construction of pPTA11: The pta⁺ allele was amplified from the S. enterica chromosome using primers pta5′SmaI (SEQ ID NO:14, 5′-TGT AAC CCG GGC CCA AAA GAC TGT AAC GA-3′) and pta3′XbaI (SEQ ID NO:15, 5′-TCA CCT CTA GAC CTG ACA AGG CGT TCA C-3′). The underlined sequence indicates the appropriate engineered restriction site. The resulting 2.2 kb fragment was end-treated and ligated into the Epicentre Copy Control vector pCC1 (Epicentre, Madison, Wis.). The pta⁺ allele was oriented within the multiple cloning site opposing P_{T7}. This facilitated cloning in single copy. The copy number was increased according to the manufacturer's instructions. This plasmid was then digested with SmaI and XbaI. The fragment containing the pta⁺ allele was gel extracted and ligated into the same sites of pBAD30. This plasmid was named pPTA11.

pPTA11 sequence (SEQ ID NO:16):

pPTA11 sequence (SEQ ID NO:16):
5′gctagcggagtgtatactggcttactatgttggcactgatgagggtgt
cagtgaagtgcttcatgtggcaggagaaaaaaggctgcaccggtgcgtca
gcagaatatgtgatacaggatatattccgcttcctcgctcactgactcgc
tacgctcggtcgttcgactgcggcgagcggaaatggcttacgaacggggc
ggagatttcctggaagatgccaggaagatacttaacagggaagtgagagg
gccgcggcaaagccgtttttccataggctccgcccccctgacaagcatca
cgaaatctgacgctcaaatcagtggtggcgaaacccgacaggactataaa
gataccaggcgtttccccctggcggctccctcgtgcgctctcctgttcct
gcctttcggtttaccggtgtcattccgctgttatggccgcgtttgtctca
ttccacgcctgacactcagttccgggtaggcagttcgctccaagctggac
tgtatgcacgaaccccccgttcagtccgaccgctgcgccttatccggtaa
ctatcgtcttgagtccaacccggaaagacatgcaaaagcaccactggcag
cagccactggtaattgatttagaggagttagtcttgaagtcatgcgccgg
ttaaggctaaactgaaaggacaagttttggtgactgcgctcctccaagcc
agttacctcggttcaaagagttggtagctcagagaaccttcgaaaaaccg
ccctgcaaggcggttttttcgttttcagagcaagagattacgcgcagacc
aaaacgatctcaagaagatcatcttattaatcagataaaatatttgctca
tgagcccgaagtggcgagcccgatcttccccatcggtgatgtcggcgata
taggcgccagcaaccgcacctgtggcgccggtgatgccggccacgatgcg
tccggcgtagaggatctgctcatgtttgacagcttatcatcgatgcataa
tgtgcctgtcaaatggacgaagcagggattctgcaaaccctatgctactc
cgtcaagccgtcaattgtctgattcgttaccaattatgacaacttgacgg
ctacatcattcactttttcttcacaaccggcacggaactcgctcgggctg
gccccggtgcattttttaaatacccgcgagaaatagagttgatcgtcaaa
accaacattgcgaccgacggtggcgataggcatccgggtggtgctcaaaa
gcagcttcgcctggctgatacgttggtcctcgcgccagcttaagacgcta
atccctaactgctggcggaaaagatgtgacagacgcgacggcgacaagca
aacatgctgtgcgacgctggcgatatcaaaattgctgtctgccaggtgat
cgctgatgtactgacaagcctcgcgtacccgattatccatcggtggatgg
agcgactcgttaatcgcttccatgcgccgcagtaacaattgctcaagcag
atttatcgccagcagctccgaatagcgcccttccccttgcccggcgttaa
tgatttgcccaaacaggtcgctgaaatgcggctggtgcgcttcatccggg
cgaaagaaccccgtattggcaaatattgacggccagttaagccattcatg
ccagtaggcgcgcggacgaaagtaaacccactggtgataccattcgcgag
cctccggatgacgaccgtagtgatgaatctctcctggcgggaacagcaaa
atatcacccggtcggcaaacaaattctcgtccctgatttttcaccacccc
ctgaccgcgaatggtgagattgagaatataacctttcattcccagcggtc
ggtcgataaaaaaatcgagataaccgttggcctcaatcggcgttaaaccc
gccaccagatgggcattaaacgagtatcccggcagcaggggatcattttg
cgcttcagccatacttttcatactcccgccattcagagaagaaaccaatt
gtccatattgcatcagacattgccgtcactgcgtcttttactggctcttc
tcgctaaccaaaccggtaaccccgcttattaaaagcattctgtaacaaag
cgggaccaaagccatgacaaaaacgcgtaacaaaagtgtctataatcacg
gcagaaaagtccacattgattatttgcacggcgtcacactttgctatgcc
atagcatttttatccataagattagcggatcctacctgacgctttttatc
gcaactctctactgtttctccatacccgtttttttgggctagcgaattcg
agctcggta (Salmonella sequence starts next) cccgGG
cccaaaagacggtaacgaaagaggataaacc gtg (start codon)
tcccgtattattatgctgatccctaccggaaccagcgtcggcctgaccag
cgtcagcctcggcgtcatccgtgctatggaacgcaaaggcgttcgtctga
gcgtctttaagcctatcgcccagcctcgcgctggcggcgatgcgcctgac
cagaccaccactatcgttcgcgcgaactctaccctgccggcggctgaacc
gctgaagatgagccacgttgaatctctgctctccagcaaccagaaagacg
tgctgatggaagagatcatcgcgaactaccatgcgaataccaaagacgcg
gaagtggtgctggttgaaggtctggttccgacccgtaaacatcagttcgc
tcagtctctgaactatgaaatcgcgaaaacgctgaatgcggaaatcgttt
ttgtcatgtctcagggtaccgacacgccagaacagctgaacgagcgtatc
gaactgacgcgcagcagcttcggcggcgcgaaaaacaccaacatcaccgg
tgttattatcaacaaactgaatgcgccggttgatgaacaaggccgtactc
gcccggatctgtcggagatctttgacgactcttccaaagcgcaggtgatc
aaaatcgatcctgctaaactgcaggaatccagcccgctgccggttctggg
cgcggtgccgtggagcttcgacctgattgcgacccgcgctatcgatatgg
cgcgtcacctgaacgccaccatcattaacgaaggcgatatcaaaacccgc
cgcgttaaatccgtcactttctgcgcgcgtagtattccgcatatgctgga
acatttccgcgcaggctcgttgttagtgacctccgctgaccgtccggacg
tactggtcgcagcttgcctggccgcgatgaacggcgtagaaatcggcgcc
ctgttgctgaccggcggctatgaaatggacgcgcgcatttctaactgtgc
gaacgcgcattcgccaccggtctgccggtctttatggtgaacaccaacac
ctggcagacttcgctgagcctgcaaagcttcaatctggaagtcccggttg
atgaccatgagcgcatcgagaaagttcaggaatacgtcgcgaactacgtt
aacgctgagtggatcgagtcgctgaccgccacttccgagcgtagccgtcg
tctctctccgccggcgttccgctaccaactgactgagctggcgcgtaaag
ccggtaaacgcgtagtgctgccggaaggcgacgaaccgcgtaccgtcaaa
gcggcggcaatctgcgctgaacgcggcatcgccacttgcgtactgctggg
caacccggatgaaatcaaccgcgtcgcggcatctcagggcgttgagctgg
gcgcaggtattgaaatcgtcgatccggaagtggtgcgtgaaagctatgtc
gctcgcctggttgagctgcgtaagagcaaaggcatgaccgaaccggttgc
ccgcgaacagctggaagacaacgtggtgctcggcacgctgatgctggagc
aagacgaagtcgacggcctggtttccggcgcggttcataccaccgccaac
accatccgtccgccgctgcagcttattaaaacggcgccgggtagctccct
ggtctcttctgtgttctttatgctactgccggaacaggtttacgtttacg
gcgactgcgcaatcaacccagacccgaccgcagagcagctggcagaaatc
gcgattcagtctgcggattccgccattgccttcggcatcgaaccgcgtgt
ggcaatgctctcctactccaccggcacctctggcgcgggcagcgacgtag
agaaagtacgtgaagcgacgcgtctggcgcaggaaaaacgtcctgacctg
atgatcgacggtccgttgcagtacgacgccgcggtcatggctgacgtagc
gaaatccaaagcgccgaactcgccggttgcgggtcgcgctaccgtgttca
ttttcccggatctgaacaccggtaacaccacctacaaagcggtacagcgt
tctgccgacctgatctccatcgggccgatgttgcagggtatgcgcaagcc
ggtgaacgacctgtcccgtggcgcgctggttgacgatatcgtctacacca
tcgccctgacggcgatccaggcttctcagcagcagcag taa (stop
codon) cagtaaaagctaatgccggatggcggcgtgaacgccttgtccg
gTctaGagtcgacctgcaggcatgcaag (Salmonella sequence
ends) cttggctgttttggcggatgagagaagattttcagcctgataca
gattaaatcagaacgcagaagcggtctgataaaacagaatttgcctggcg
gcagtagcgcggtggtcccacctgaccccatgccgaactcagaagtgaaa
cgccgtagcgccgatggtagtgtggggtctccccatgcgagagtagggaa
ctgccaggcatcaaataaaacgaaaggctcagtcgaaagactgggccttt
cgttttatctgttgtttgtcggtgaacgctctcctgagtaggacaaatcc
gccgggagcggatttgaacgttgcgaagcaacggcccggagggtggcggg
caggacgcccgccataaactgccaggcatcaaattaagcagaaggccatc
ctgacggatggcctttttgcgtttctacaaactcttttgtttatttttct
aaatacattcaaatatgtatccgctcatgagacaataaccctgataaatg
cttcaataatattgaaaaaggaagagtatgagtattcaacatttccgtgt
cgcccttattcccttttttgcggcattttgccttcctgtttttgctcacc
cagaaacgctggtgaaagtaaaagatgctgaagatcagttgggtgcacga
gtgggttacatcgaactggatctcaacagcggtaagatccttgagagttt
tcgccccgaagaacgttttccaatgatgagcacttttaaagttctgctat
gtggcgcggtattatcccgtgttgacgccgggcaagagcaactcggtcgc
cgcatacactattctcagaatgacttggttgagtactcaccagtcacaga
aaagcatcttacggatggcatgacagtaagagaattatgcagtgctgcca
taaccatgagtgataacactgcggccaacttacttctgacaacgatcgga
ggaccgaaggagctaaccgcttttttgcacaacatgggggatcatgtaac
tcgccttgatcgttgggaaccggagctgaatgaagccataccaaacgacg
agcgtgacaccacgatgcctgcagcaatggcaacaacgttgcgcaaacta
ttaactggcgaactacttactctagcttcccggcaacaattaatagactg
gatggaggcggataaagttgcaggaccacttctgcgctcggcccttccgg
ctggctggtttattgctgataaatctggagccggtgagcgtgggtctcgc
ggtatcattgcagcactggggccagatggtaagccctcccgtatcgtagt
tatctacacgacggggagtcaggcaactatggatgaacgaaatagacaga
tcgctgagataggtgcctcactgattaagcattggtaactgtcagaccaa
gtttactcatatatactttagattgatttacgcgccctgtagcggcgcat
taagcgcggcgggtgtggtggttacgcgcagcgtgaccgctacacttgcc
agcgccctagcgcccgctcctttcgctttcttcccttcctttctcgccac
gttcgccggctttccccgtcaagctctaaatcgggggctccctttagggt
tccgatttagtgctttacggcacctcgaccccaaaaaacttgatttgggt
gatggttcacgtagtgggccatcgccctgatagacggtttttcgcccttt
gacgttggagtccacgttctttaatagtggactcttgttccaaacttgaa
caacactcaaccctatctcgggctattcttttgatttataagggattttg
ccgatttcggcctattggttaaaaaatgagctgatttaacaaaaatttaa
cgcgaattttaacaaaatattaacgtttacaatttaaaaggatctaggtg
aagatcctttttgataatctcatgaccaaaatcccttaacgtgagttttc
gttccactgagcgtcagaccccgtagaaaagatcaaaggatcttcttgag
atcctttttttctgcgcgtaatctgctgcttgcaaacaaaaaaaccaccg
ctaccagcggtggtttgtttgccggatcaagagctaccaactctttttcc
gaaggtaactggcttcagcagagcgcagataccaaatactgtccttctag
tgtagccgtagttaggccaccacttcaagaactctgtagcaccgcctaca
tacctcgctctgctaatcctgttaccagtcaggcatttgagaagcacacg
gtcacactgcttccggtagtcaataaaccggtaaaccagcaatagacata
agcggctatttaacgaccctgccctgaaccgacgaccgggtcgaatttgc
tttcgaatttctgccattcatccgcttattatcacttattcaggcgtagc
accaggcgtttaagggcaccaataactgccttaaaaaaattacgccccgc
cctgccactcatcgcagtactgttgtaattcattaagcattctgccgaca
tggaagccatcacagacggcatgatgaacctgaatcgccagcggcatcag
caccttgtcgccttgcgtataatatttgcccatg3′

### EXAMPLE 2

### Plasmids for Producing Pta

This is an example on how to construct a plasmid that can be transformed into bacteria for producing Pta. Although only the production of wild-type S. enterica Pta is used as an example, it is understood that other proteins of interest such as Pta's from other bacterial species and the N-terminal domain of a Pta can be produced similarly.

Construction of pPTA14: pta⁺ from S. enterica was amplified from pPTA11 (example 1) using SeptaN5′NcoI (SEQ ID NO:17, 5′-GAG GAT AAA CCA TGG CCC GTA TTA TTA TGC TG-3′) and pta3′XbaI (SEQ ID NO:18, 5′-TCA CCT CTA GAC CTG ACA AGG CGT TCA C-3′). The underlined sequence indicates the location of the appropriate restriction site. The SeptaN5′NcoI primer converted the GTG start site to an ATG start site and engineered the mutation S2A as a result of using the NcoI restriction site. The 2.2 kb pta⁺ fragment was A-tailed, gel purified, and ligated into the MCS of pGEM-T-Easy (Promega, Madison, Wis.). pta⁺ was oriented opposing P_{lacZ}. This plasmid was then digested with NcoI and EcoRI and the fragment containing pta⁺ was ligated into the same site of pET42-A (Novagen, Madison, Wis.). This plasmid was named pPTA14 (Fuses N-terminal GST-(His)₆-S-tag to Pta).

pPTA14 sequence (SEQ ID NO:19):

pPTA14 sequence (SEQ ID NO:19):
5′tggcgaatgggacgcgccctgtagcggcgcattaagcgcggcgggtgt
ggtggttacgcgcagcgtgaccgctacacttgccagcgccctagcgcccg
ctcctttcgctttcttcccttcctttctcgccacgttcgccggctttccc
cgtcaagctctaaatcgggggctccctttagggttccgatttagtgcttt
acggcacctcgaccccaaaaaacttgattagggtgatggttcacgtagtg
ggccatcgccctgatagacggtttttcgccctttgacgttggagtccacg
ttctttaatagtggactcttgttccaaactggaacaacactcaaccctat
ctcggtctattcttttgatttataagggattttgccgatttcggcctatt
ggttaaaaaatgagctgatttaacaaaaatttaacgcgaattttaacaaa
atattaacgtttacaatttcaggtggcacttttcggggaaatgtgcgcgg
aacccctatttgtttatttttctaaatacattcaaatatgtatccgctca
tgaattaattcttagaaaaactcatcgagcatcaaatgaaactgcaattt
attcatatcaggattatcaataccatatttttgaaaaagccgtttctgta
atgaaggagaaaactcaccgaggcagttccataggatggcaagatcctgg
tatcggtctgcgattccgactcgtccaacatcaatacaacctattaattt
cccctcgtcaaaaataaggttatcaagtgagaaatcaccatgagtgacga
ctgaatccggtgagaatggcaaaagtttatgcatttctttccagacttgt
tcaacaggccagccattacgctcgtcatcaaaatcactcgcatcaaccaa
accgttattcattcgtgattgcgcctgagcgagacgaaatacgcgatcgc
tgttaaaaggacaattacaaacaggaatcgaatgcaaccggcgcaggaac
actgccagcgcatcaacaatattttcacctgaatcaggatattcttctaa
tacctggaatgctgttttcccggggatcgcagtggtgagtaaccatgcat
catcaggagtacggataaaatgcttgatggtcggaagaggcataaattcc
gtcagccagtttagtctgaccatctcatctgtaacatcattggcaacgct
acctttgccatgtttcagaaacaactctggcgcatcgggcttcccataca
atcgatagattgtcgcacctgattgcccgacattatcgcgagcccattta
tacccatataaatcagcatccatgttggaatttaatcgcggcctagagca
agacgtttcccgttgaatatggctcataacaccccttgtattactgttta
tgtaagcagacagttttattgttcatgaccaaaatcccttaacgtgagtt
ttcgttccactgagcgtcagaccccgtagaaaagatcaaaggatcttctt
gagatcctttttttctgcgcgtaatctgctgcttgcaaacaaaaaaacca
ccgctaccagcggtggtttgtttgccggatcaagagctaccaactctttt
tccgaaggtaactggcttcagcagagcgcagataccaaatactgtccttc
tagtgtagccgtagttaggccaccacttcaagaactctgtagcaccgcct
acatacctcgctctgctaatcctgttaccagtggctgctgccagtggcga
taagtcgtgtcttaccgggttggactcaagacgatagttaccggataagg
cgcagcggtcgggctgaacggggggttcgtgcacacagcccagcttggag
cgaacgacctacaccgaactgagatacctacagcgtgagctatgagaaag
cgccacgcttcccgaagggagaaaggcggacaggtatccggtaagcggca
gggtcggaacaggagagcgcacgagggagcttccagggggaaacgcctgg
tatctttatagtcctgtcgggtttcgccacctctgacttgagcgtcgatt
tttgtgatgctcgtcaggggggcggagcctatggaaaaacgccagcaacg
cggcctttttacggttcctggccttttgctggccttttgctcacatgttc
tttcctgcgttatcccctgattctgtggataaccgtattaccgcctttga
gtgagctgataccgctcgccgcagccgaacgaccgagcgcagcgagtcag
tgagcgaggaagcggaagagcgcctgatgcggtattttctccttacgcat
ctgtgcggtatttcacaccgcatatatggtgcactctcagtacaatctgc
tctgatgccgcatagttaagccagtatacactccgctatcgctacgtgac
tgggtcatggctgcgccccgacacccgccaacacccgctgacgcgccctg
acgggcttgtctgctcccggcatccgcttacagacaagctgtgaccgtct
ccgggagctgcatgtgtcagaggttttcaccgtcatcaccgaaacgcgcg
aggcagctgcggtaaagctcatcagcgtggtcgtgaagcgattcacagat
gtctgcctgttcatccgcgtccagctcgttgagtttctccagaagcgtta
atgtctggcttctgataaagcgggccatgttaagggcggttttttcctgt
ttggtcactgatgcctccgtgtaagggggatttctgttcatgggggtaat
gataccgatgaaacgagagaggatgctcacgatacgggttactgatgatg
aacatgcccggttactggaacgttgtgagggtaaacaactggcggtatgg
atgcggcgggaccagagaaaaatcactcagggtcaatgccagcgcttcgt
taatacagatgtaggtgttccacagggtagccagcagcatcctgcgatgc
agatccggaacataatggtgcagggcgctgacttccgcgtttccagactt
tacgaaacacggaaaccgaagaccattcatgttgttgctcaggtcgcaga
cgttttgcagcagcagtcgcttcacgttcgctcgcgtatcggtgattcat
tctgctaaccagtaaggcaaccccgccagcctagccgggtcctcaacgac
aggagcacgatcatgctagtcatgccccgcgcccaccggaaggagctgac
tgggttgaaggctctcaagggcatcggtcgagatcccggtgcctaatgag
tgagctaacttacattaattgcgttgcgctcactgcccgctttccagtcg
ggaacctgtcgtgccagctgcattaatgaatcggccaacgcgcggggaga
ggcggtttgcgtattgggcgccagggtggtttttcttttcaccagtgaga
cgggcaacagctgattgcccttcaccgcctggccctgagagagttgcagc
aagcggtccacgctggtttgccccagcaggcgaaaatcctgtttgatggt
ggttaacggcgggatataacatgagctgtcttcggtatcgtcgtatccca
ctaccgagatgtccgcaccaacgcgcagcccggactcggtaatggcgcgc
attgcgcccagcgccatctgatcgttggcaaccagcatcgcagtgggaac
gatgccctcattcagcatttgcatggtttgttgaaaaccggacatggcac
tccagtcgccttcccgttccgctatcggctgaatttgattgcgagtgaga
tatttatgccagccagccagacgcagacgcgccgagacagaacttaatgg
gcccgctaacagcgcgatttgctggtgacccaatgcgaccagatgctcca
cgcccagtcgcgtaccgtcttcatgggagaaaataatactgttgatgggt
gtctggtcagagacatcaagaaataacgccggaacattagtgcaggcagc
ttccacagcaatggcatcctggtcatccagcggatagttaatgatcagcc
cactgacgcgttgcgcgagaagattgtgcaccgccgctttacaggcttcg
acgccgcttcgttctaccatcgacaccaccacgctggcacccagttgatc
ggcgcgagatttaatcgccgcgacaatttgcgacggcgcgtgcagggcca
gactggaggtggcaacgccaatcagcaacgactgtttgcccgccagttgt
tgtgccacgcggttgggaatgtaattcagctccgccatcgccgcttccac
tttttcccgcgttttcgcagaaacgtggctggcctggttcaccacgcggg
aaacggtctgataagagacaccggcatactctgcgacatcgtataacgtt
actggtttcacattcaccaccctgaattgactctcttccgggcgctatca
tgccataccgcgaaaggttttgcgccattcgatggtgtccgggatctcga
cgctctcccttatgcgactcctgcattaggaagcagcccagtagtaggtt
gaggccgttgagcaccgccgccgcaaggaatggtgcatgcaaggagatgg
cgcccaacagtcccccggccacggggcctgccaccatacccacgccgaaa
caagcgctcatgagcccgaagtggcgagcccgatcttccccatcggtgat
gtcggcgatataggcgccagcaaccgcacctgtggcgccggtgatgccgg
ccacgatgcgtccggcgtagaggatcgagatcgatctcgatcccgcgaaa
ttaatacgactcactataggggaattgtgagcggataacaattcccctct
agaaataattttgtttaactttaagaaggagatatacatatgtcccctat
actaggttattggaaaattaagggccttgtgcaacccactcgacttcttt
tggaatatcttgaagaaaaatatgaagagcatttgtatgagcgcgatgaa
ggtgataaatggcgaaacaaaaagtttgaattgggtttggagtttcccaa
tcttccttattatattgatggtgatgttaaattaacacagtctatggcca
tcatacgttatatagctgacaagcacaac atg (fusion protein
start codon) ttgggtggttgtccaaaagagcgtgcagagatttcaa
tgcttgaaggagcggttttggatattagatacggtgtttcgagaattgca
tatagtaaagactttgaaactctcaaagttgattttcttagcaagctacc
tgaaatgctgaaaatgttcgaagatcgtttatgtcataaaacatatttaa
atggtgatcatgtaacccatcctgacttcatgttgtatgacgctcttgat
gttgttttatacatggacccaatgtgcctggatgcgttcccaaaattagt
ttgttttaaaaacgtattgaagctatcccacaaattgataagtacttgaa
atccagcaagtatatagcatggcctttgcagggctggcaagccacgtttg
gtggtggcgaccatcctccaaatcggatggttcaactagtggttctggtc
atcaccatcaccatcactccgcgggtctggtgccacgcggtagtactgca
attggtatgaaagaaaccgctgctgctaaattcgaacgccagcacatgga
cagcccagatctgggtaccggtggtggctccggtattgagggacgcgggt
cc (S. enterica sequence starts next with GTGT →
ATGG: V1M and S2A mutations) atg G cccgtattattatgc
tgatccctaccggaaccagcgtcggcctgaccagcgtcagcctcggcgtc
atccgtgctatggaacgcaaaggcgttcgtctgagcgtctttaagcctat
cgcccagcctcgcgctggcggcgatgcgcctgaccagaccaccactatcg
ttcgcgcgaactctaccctgccggcggctgaaccgctgaagatgagccac
gttgaatctctgctctccagcaaccagaaagacgtgctgatggaagagat
catcgcgaactaccatgcgaataccaaagacgcggaagtggtgctggttg
aaggtctggttccgacccgtaaacatcagttcgctcagtctctgaactat
gaaatcgcgaaaacgctgaatgcggaaatcgtttttgtcatgtctcaggg
taccgacacgccagaacagctgaacgagcgtatcgaactgacgcgcagca
gcttcggcggcgcgaaaaacaccaacatcaccggtgttattatcaacaaa
ctgaatgcgccggttgatgaacaaggccgtactcgcccggatctgtcgga
gatctttgacgactcttccaaagcgcaggtgatcaaaatcgatcctgcta
aactgcaggaatccagcccgctgccggttctgggcgcggtgccgtggagc
ttcgacctgattgcgacccgcgctatcgatatggcgcgtcacctgaacgc
caccatcattaacgaaggcgatatcaaaacccgccgcgttaaatccgtca
ctttctgcgcgcgtagtattccgcatatgctggaacatttccgcgcaggc
tcgttgttagtgacctccgctgaccgtccggacgtactggtcgcagcttg
cctggccgcgatgaacggcgtagaaatcggcgccctgttgctgaccggcg
gctatgaaatggacgcgcgcatttctaaactgtgcgaacgcgcattcgcc
accggtctgccggtctttatggtgaacaccaacacctggcagacttcgct
gagcctgcaaagcttcaatctggaagtcccggttgatgaccatgagcgca
tcgagaaagttcaggaatacgtcgcgaactacgttaacgctgagtggatc
gagtcgctgaccgccacttccgagcgtagccgtcgtctctctccgccggc
gttccgctaccaactgactgagctggcgcgtaaagccggtaaacgcgtag
tgctgccggaaggcgacgaaccgcgtaccgtcaaagcggcggcaatctgc
gctgaacgcggcatcgccacttgcgtactgctgggcaacccggatgaaat
caaccgcgtcgcggcatctcagggcgttgagctgggcgcaggtattgaaa
tcgtcgatccggaagtggtgcgtgaaagctatgtcgctcgcctggttgag
ctgcgtaagagcaaaggcatgaccgaaccggttgcccgcgaacagctgga
agacaacgtggtgctcggcacgctgatgctggagcaagacgaagtcgacg
gcctggtttccggcgcggttcataccaccgccaacaccatccgtccgccg
ctgcagcttattaaaacggcgccgggtagctccctggtctcttctgtgtt
ctttatgctactgccggaacaggtttacgtttacggcgactgcgcaatca
acccagacccgaccgcagagcagctggcagaaatcgcgattcagtctgcg
gattccgccattgccttcggcatcgaaccgcgtgtggcaatgctctccta
ctccaccggcacctctggcgcgggcagcgacgtagagaaagtacgtgaag
cgacgcgtctggcgcaggaaaaacgtcctgacctgatgatcgacggtccg
ttgcagtacgacgccgcggtcatggctgacgtagcgaaatccaaagcgcc
gaactcgccggttgcgggtcgcgctaccgtgttcattttcccggatctga
acaccggtaacaccacctacaaagcggtacagcgttctgccgacctgatc
tccatcgggccgatgttgcagggtatgcgcaagccggtgaacgacctgtc
ccgtggcgcgctggttgacgatatcgtctacaccatcgccctgacggcga
tccaggcttctcagcagcagcag taa (fusion protein stop
codon) cagtaaaagctaatgccggatggcggcgtgaacgccttgtcag
gTctaGaggtgaAATCACTAGTGAATTC (end of S. enterica
sequence) tgtacaggccttggcgcgcctgcaggcgagctccgtcgac
aagcttgcggccgcactcgagcaccaccaccaccaccaccaccactaatt
gattaatacctaggctgctaaacaaagcccgaaaggaagctgagttggct
gctgccaccgctgagcaataactagcataaccccttggggcctctaaacg
ggtcttgaggggttttttgctgaaaggaggaactatatccggat3′
pPTA14 overproduced protein sequence (SEQ ID NO:
20) (* = Stop):
(N-terminal fusion tag) MSPILGYWKIKGLVQPTRLLLEYLEE
KYEEHLYERDEGDKWRNKKFELGLEFPNLPYYIDGDVKLTQSMAIIRYIA
DKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKL
VCFKKRIEAIPQIDKYLKSSKYIAWPLQGWQATFGGGDHPPKSDGSTSGS
GHHHHHHSAGLVPRGSTAIGMKETAAAKFERQHMDSPDLGTGGGSGIEGR
GS (N-terminal fusion tag ends and Pta starts
next) MaRIIMLIPTGTSVGLTSVSLGVIRAMERKGVRLSVFKPIAQPR
AGGDAPDQTTTIVRANSTLPAAEPLKMSHVESLLSSNQKDVLMEEIIANY
HANTKDAEVVLVEGLVPTRKHQFAQSLNYEIAKTLNAEIVFVMSQGTDTP
EQLNERIELTRSSFGGAKNTNITGVIINKLNAPVDEQGRTRPDLSEIFDD
SSKAQVIKIDPAKLQESSPLPVLGAVPWSFDLIATRAIDMARHLNATIIN
EGDIKTRRVKSVTFCARSIPHMLELIFRAGSLLVTSADRPDVLVAACLAA
MNGVEIGALLLTGGYEMDARIISKLCERAFATGLPVFMVNTNTWQTSLSL
QSFNLEVPVDDHERIEKVQEYVANYVNAEWIESLTATSERSRRLSPPAFR
YQLTELARKAGKRVVLPEGDEPRTVKAAAICAERGIATCVLLGNPDEINR
VAASQGVELGAGIEIVDPEVVRESYVARLVELRKSKGMTEPVAREQLEDN
VVLGTLMLEQDEVDGLVSGAVHTTANTIRPPLQLIKTAPGSSLVSSVFFM
LLPEQVYVYGDCAINPDPTAEQLAEIAIQSADSAIAFGIEPRVAMLSYST
GTSGAGSDVEKVREATRLAQEKRPDLMIDGPLQYDAAVMADVAKSKAPNS
PVAGRATVFWPDLNTGNTTYKAVQRSADLISIGPMILQGMRKPVNDLSRG
ALVDDIVYTIALTAIQASQQQQ*.

### EXAMPLE 3

### Plasmids for Genetic Analysis in Animal Models

This is an example on how to construct a plasmid that can be employed to assay the function (e.g., virulence) of wild-type and mutant alleles of pta in an animal model such as that used in example 4 below. The plasmid construction uses a variant of the pBAD30 vector with an engineered mutation in the P_{araBAD} promoter that has been shown previously to block catabolite repression (Colome, J., G. Wilcox, and E. Englesberg. 1977. Constitutive mutations in the controlling site region of the araBAD operon of Escherichia coli B/r that decrease sensitivity to catabolite repression. J. Bacterol. 129:948-958; and Horwitz, A. H., C. Morandi, and G. Wilcox. 1980. Deoxyribonucleic acid sequence of araBAD promoter mutants of Escherichia coli. J. Bacterol. 142:659-667).

Construction of pPTA15: pBAD30 was subjected to site directed mutagenesis using Stratagene's Quick Change kit (Stratagene, La Jolla, Calif.) using mutagenic primers araX^{C}5′QC (SEQ ID NO:21, 5′-TCG CAA CTC TCT ACT ATT TCT CCA TAC CCG-3′) and araX^{C}3′QC (SEQ ID NO:22, 5′-CGG GTA TGG AGA AAT AGT AGA GAG TTG CGA-3′) (mutagenic base underlined). This araX^{C} mutation was characterized previously to severely block catabolite repression of the P_{araBAD} promoter (Horwitz, A. H., C. Morandi, and G. Wilcox. 1980. Deoxyribonucleic acid sequence of araBAD promoter mutants of Escherichia coli. J. Bacterol. 142:659-667). This plasmid was confirmed by sequencing and was named pBAD30 araX^{C}. The pta+ allele was cut out of pPTA11 using EcoRI and XbaI and was ligated into the same sites of pBAD30 araX^{C}. This plasmid was named pPTA15.

pPTA15 sequence (SEQ ID NO:23) (same as pPTA11, except for araX^{C} mutation at bp 2262 indicated by capitalized, larger font “A”):

pPTA15 sequence (SEQ ID NO:23) (same as pPTA11,
except for araX^{C} mutation at bp 2262 indicated by
capitalized, larger font “A”):
5′gctagcggagtgtatactggcttactatgttggcactgatgagggtgt
cagtgaagtgcttcatgtggcaggagaaaaaaggctgcaccggtgcgtca
gcagaatatgtgatacaggatatattccgcttcctcgctcactgactcgc
tacgctcggtcgttcgactgcggcgagcggaaatggcttacgaacggggc
ggagatttcctggaagatgccaggaagatacttaacagggaagtgagagg
gccgcggcaaagccgtttttccataggctccgcccccctgacaagcatca
cgaaatctgacgctcaaatcagtggtggcgaaacccgacaggactataaa
gataccaggcgtttccccctggcggctccctcgtgcgctctcctgttcct
gcctttcggtttaccggtgtcattccgctgttatggccgcgtttgtctca
ttccacgcctgacactcagttccgggtaggcagttcgctccaagctggac
tgtatgcacgaaccccccgttcagtccgaccgctgcgccttatccggtaa
ctatcgtcttgagtccaacccggaaagacatgcaaaagcaccactggcag
cagccactggtaattgatttagaggagttagtcttgaagtcatgcgccgg
ttaaggctaaactgaaaggacaagttttggtgactgcgctcctccaagcc
agttacctcggttcaaagagttggtagctcagagaaccttcgaaaaaccg
ccctgcaaggcggttttttcgttttcagagcaagagattacgcgcagacc
aaaacgatctcaagaagatcatcttattaatcagataaaatatttgctca
tgagcccgaagtggcgagcccgatcttccccatcggtgatgtcggcgata
taggcgccagcaaccgcacctgtggcgccggtgatgccggccacgatgcg
tccggcgtagaggatctgctcatgtttgacagcttatcatcgatgcataa
tgtgcctgtcaaatggacgaagcagggattctgcaaaccctatgctactc
cgtcaagccgtcaattgtctgattcgttaccaattatgacaacttgacgg
ctacatcattcactttttcttcacaaccggcacggaactcgctcgggctg
gccccggtgcattttttaaatacccgcgagaaatagagttgatcgtcaaa
accaacattgcgaccgacggtggcgataggcatccgggtggtgctcaaaa
gcagcttcgcctggctgatacgttggtcctcgcgccagcttaagacgcta
atccctaactgctggcggaaaagatgtgacagacgcgacggcgacaagca
aacatgctgtgcgacgctggcgatatcaaaattgctgtctgccaggtgat
cgctgatgtactgacaagcctcgcgtacccgattatccatcggtggatgg
agcgactcgttaatcgcttccatgcgccgcagtaacaattgctcaagcag
atttatcgccagcagctccgaatagcgcccttccccttgcccggcgttaa
tgatttgcccaaacaggtcgctgaaatgcggctggtgcgcttcatccggg
cgaaagaaccccgtattggcaaatattgacggccagttaagccattcatg
ccagtaggcgcgcggacgaaagtaaacccactggtgataccattcgcgag
cctccggatgacgaccgtagtgatgaatctctcctggcgggaacagcaaa
atatcacccggtcggcaaacaaattctcgtccctgatttttcaccacccc
ctgaccgcgaatggtgagattgagaatataacctttcattcccagcggtc
ggtcgataaaaaaatcgagataaccgttggcctcaatcggcgttaaaccc
gccaccagatgggcattaaacgagtatcccggcagcaggggatcattttg
cgcttcagccatacttttcatactcccgccattcagagaagaaaccaatt
gtccatattgcatcagacattgccgtcactgcgtcttttactggctcttc
tcgctaaccaaaccggtaaccccgcttattaaaagcattctgtaacaaag
cgggaccaaagccatgacaaaaacgcgtaacaaaagtgtctataatcacg
gcagaaaagtccacattgattatttgcacggcgtcacactttgctatgcc
atagcatttttatccataagattagcggatcctacctgacgctttttatc
gcaactctctactAtttctccatacccgtttttttgggctagcgaattcg
agctcggta (Salmonella sequence starts next) cccgGG
cccaaaagacggtaacgaaagaggataaacc gtg (start codon)
tcccgtattattatgctgatccctaccggaaccagcgtcggcctgaccag
cgtcagcctcggcgtcatccgtgctatggaacgcaaaggcgttcgtctga
gcgtctttaagcctatcgcccagcctcgcgctggcggcgatgcgcctgac
cagaccaccactatcgttcgcgcgaactctaccctgccggcggctgaacc
gctgaagatgagccacgttgaatctctgctctccagcaaccagaaagacg
tgctgatggaagagatcatcgcgaactaccatgcgaataccaaagacgcg
gaagtggtgctggttgaaggtctggttccgacccgtaaacatcagttcgc
tcagtctctgaactatgaaatcgcgaaaacgctgaatgcggaaatcgttt
ttgtcatgtctcagggtaccgacacgccagaacagctgaacgagcgtatc
gaactgacgcgcagcagcttcggcggcgcgaaaaacaccaacatcaccgg
tgttattatcaacaaactgaatgcgccggttgatgaacaaggccgtactc
gcccggatctgtcggagatctttgacgactcttccaaagcgcaggtgatc
aaaatcgatcctgctaaactgcaggaatccagcccgctgccggttctggg
cgcggtgccgtggagcttcgacctgattgcgacccgcgctatcgatatgg
cgcgtcacctgaacgccaccatcattaacgaaggcgatatcaaaacccgc
cgcgttaaatccgtcactttctgcgcgcgtagtattccgcatatgctgga
acatttccgcgcaggctcgttgttagtgacctccgctgaccgtccggacg
tactggtcgcagcttgcctggccgcgatgaacggcgtagaaatcggcgcc
ctgttgctgaccggcggctatgaaatggacgcgcgcatttctaaactgtg
cgaacgcgcattcgccaccggtctgccggtctttatggtgaacaccaaca
cctggcagacttcgctgagcctgcaaagcttcaatctggaagtcccggtt
gatgaccatgagcgcatcgagaaagttcaggaatacgtcgcgaactacgt
taacgctgagtggatcgagtcgctgaccgccacttccgagcgtagccgtc
gtctctctccgccggcgttccgctaccaactgactgagctggcgcgtaaa
gccggtaaacgcgtagtgctgccggaaggcgacgaaccgcgtaccgtcaa
agcggcggcaatctgcgctgaacgcggcatcgccacttgcgtactgctgg
gcaacccggatgaaatcaaccgcgtcgcggcatctcagggcgttgagctg
ggcgcaggtattgaaatcgtcgatccggaagtggtgcgtgaaagctatgt
cgctcgcctggttgagctgcgtaagagcaaaggcatgaccgaaccggttg
cccgcgaacagctggaagacaacgtggtgctcggcacgctgatgctggag
caagacgaagtcgacggcctggtttccggcgcggttcataccaccgccaa
caccatccgtccgccgctgcagcttattaaaacggcgccgggtagctccc
tggtctcttctgtgttctttatgctactgccggaacaggtttacgtttac
ggcgactgcgcaatcaacccagacccgaccgcagagcagctggcagaaat
cgcgattcagtctgcggattccgccattgccttcggcatcgaaccgcgtg
tggcaatgctctcctactccaccggcacctctggcgcgggcagcgacgta
gagaaagtacgtgaagcgacgcgtctggcgcaggaaaaacgtcctgacct
gatgatcgacggtccgttgcagtacgacgccgcggtcatggctgacgtag
cgaaatccaaagcgccgaactcgccggttgcgggtcgcgctaccgtgttc
attttcccggatctgaacaccggtaacaccacctacaaagcggtacagcg
ttctgccgacctgatctccatcgggccgatgttgcagggtatgcgcaagc
cggtgaacgacctgtcccgtggcgcgctggttgacgatatcgtctacacc
atcgccctgacggcgatccaggcttctcagcagcagcag taa (stop
codon) cagtaaaagctaatgccggatggcggcgtgaacgccttgtccg
gTctaGagtcgacctgcaggcatgcaag (Salmonella sequence
ends) cttggctgttttggcggatgagagaagattttcagcctgataca
gattaaatcagaacgcagaagcggtctgataaaacagaatttgcctggcg
gcagtagcgcggtggtcccacctgaccccatgccgaactcagaagtgaaa
cgccgtagcgccgatggtagtgtggggtctccccatgcgagagtagggaa
ctgccaggcatcaaataaaacgaaaggctcagtcgaaagactgggccttt
cgttttatctgttgtttgtcggtgaacgctctcctgagtaggacaaatcc
gccgggagcggatttgaacgttgcgaagcaacggcccggagggtggcggg
caggacgcccgccataaactgccaggcatcaaattaagcagaaggccatc
ctgacggatggcctttttgcgtttctacaaactcttttgtttatttttct
aaatacattcaaatatgtatccgctcatgagacaataaccctgataaatg
cttcaataatattgaaaaaggaagagtatgagtattcaacatttccgtgt
cgcccttattcccttttttgcggcattttgccttcctgtttttgctcacc
cagaaacgctggtgaaagtaaaagatgctgaagatcagttgggtgcacga
gtgggttacatcgaactggatctcaacagcggtaagatccttgagagttt
tcgccccgaagaacgttttccaatgatgagcacttttaaagttctgctat
gtggcgcggtattatcccgtgttgacgccgggcaagagcaactcggtcgc
cgcatacactattctcagaatgacttggttgagtactcaccagtcacaga
aaagcatcttacggatggcatgacagtaagagaattatgcagtgctgcca
taaccatgagtgataacactgcggccaacttacttctgacaacgatcgga
ggaccgaaggagctaaccgcttttttgcacaacatgggggatcatgtaac
tcgccttgatcgttgggaaccggagctgaatgaagccataccaaacgacg
agcgtgacaccacgatgcctgcagcaatggcaacaacgttgcgcaaacta
ttaactggcgaactacttactctagcttcccggcaacaattaatagactg
gatggaggcggataaagttgcaggaccacttctgcgctcggcccttccgg
ctggctggtttattgctgataaatctggagccggtgagcgtgggtctcgc
ggtatcattgcagcactggggccagatggtaagccctcccgtatcgtagt
tatctacacgacggggagtcaggcaactatggatgaacgaaatagacaga
tcgctgagataggtgcctcactgattaagcattggtaactgtcagaccaa
gtttactcatatatactttagattgatttacgcgccctgtagcggcgcat
taagcgcggcgggtgtggtggttacgcgcagcgtgaccgctacacttgcc
agcgccctagcgcccgctcctttcgctttcttcccttcctttctcgccac
gttcgccggctttccccgtcaagctctaaatcgggggctccctttagggt
tccgatttagtgctttacggcacctcgaccccaaaaaacttgatttgggt
gatggttcacgtagtgggccatcgccctgatagacggtttttcgcccttt
gacgttggagtccacgttctttaatagtggactcttgttccaaacttgaa
caacactcaaccctatctcgggctattcttttgatttataagggattttg
ccgatttcggcctattggttaaaaaatgagctgatttaacaaaaatttaa
cgcgaattttaacaaaatattaacgtttacaatttaaaaggatctaggtg
aagatcctttttgataatctcatgaccaaaatcccttaacgtgagttttc
gttccactgagcgtcagaccccgtagaaaagatcaaaggatcttcttgag
atcctttttttctgcgcgtaatctgctgcttgcaaacaaaaaaaccaccg
ctaccagcggtggtttgtttgccggatcaagagctaccaactctttttcc
gaaggtaactggcttcagcagagcgcagataccaaatactgtccttctag
tgtagccgtagttaggccaccacttcaagaactctgtagcaccgcctaca
tacctcgctctgctaatcctgttaccagtcaggcatttgagaagcacacg
gtcacactgcttccggtagtcaataaaccggtaaaccagcaatagacata
agcggctatttaacgaccctgccctgaaccgacgaccgggtcgaatttgc
tttcgaatttctgccattcatccgcttattatcacttattcaggcgtagc
accaggcgtttaagggcaccaataactgccttaaaaaaattacgccccgc
cctgccactcatcgcagtactgttgtaattcattaagcattctgccgaca
tggaagccatcacagacggcatgatgaacctgaatcgccagcggcatcag
caccttgtcgccttgcGtataatatttgcccatg3′

### EXAMPLE 4

### Functional Pta is Important for Bacterial Virulence in Animals

In Salmonella enterica serovar Typhimurium (hereafter referred to as S. enterica), phosphotransacetylase enzyme activity is required for the efficient metabolism of short-chain fatty acids such as acetate and propionate, both of which are abundant in the human intestine. Virulence of a pta derivative (pta::MudJ[kan⁺]) of S. enterica 14028 (Fang F C, DeGroote M A, Foster J W, Baumler A J, Ochsner U, Testerman T, Bearson S, Giard J C, Xu Y, Campbell G, and Laessig T. Virulent Salmonella typhimurium has two periplasmic Cu, Zn-superoxide dismutases. Proc Natl Acad Sci USA. 1999 Jun. 22;96(13):7502-7) was tested in BALB/c and BALB/c.D2 congenic mice. The former strain carries a point mutation in the Nramp1 gene that makes it susceptible to S. enterica infections. The latter have a wild-type Nramp1 gene from DBA/2, and these mice are about 1,000 times more resistant to S. enterica infections. 7×10³ cells of pta⁺ (S. enterica with intact pta) or pta cells (S. enterica that carried a loss-of-function pta mutation) were injected into the mice by the i.p. route. Mice were sacrificed 5 and 10 days after infection, and the ratio of pta⁺/pta bacteria was determined in livers and spleens. In repeat experiments the ratio in livers and spleens was 20-50:1 on both days, indicating that the pta mutation significantly reduced the ability of S. enterica to grow in resistant mice, even though it grew normally in LB broth. When 50-100 pta mutant bacteria were i.p. injected into BALB/c mice they died, though more slowly than the mice infected with 14028. Quantitative cultures of livers and spleens done 3 and 6 days after infection with the pta mutant showed that the mutant grew more slowly than the pta⁺ strain, but by day 6 it reached near lethal concentrations. The congenic mice were then injected with iron dextran 24 hours before they were infected with mixtures of the two strains of S. enterica. The growth of 14028 was enhanced nearly >200 fold, while the number of pta mutants was increased only 40 fold. It is concluded that the pta mutation compromises the ability of S. enterica to obtain iron in vivo when the host has a normal Nramp1 gene. Pta is less important in mice with a mutant Nramp1. This implies that Nramp1 might be involved in withholding iron from the pathogen, and that Salmonella may utilize a mechanism that requires pta to scavenge iron in vivo.

### EXAMPLE 5

### N-terminal Domain of a Long Form Pta is not Required for Pta Enzymatic Activity but Required for Virulence

Materials and Methods

All Salmonella strains used in this study were derivatives of S. enterica serovar Typhimurium LT2. S. enterica strains were grown on a minimal medium (Berkowitz, et al., J. Bacteriol. 96:215-220, 1968) supplemented with 1 mM MgSO₄ and 0.5 mM L-methionine. Nutrient Broth (NB) was the rich medium used to cultivate S. enterica strains while Luria-Bertani broth (LB) was used to cultivate E. coli strains. Genes under the control of the P_{araBAD} promoter were induced for expression by addition of L-(+)-arabinose to a final concentration of 200 μM for growth on acetate. Ampicillin, kanamycin, tetracycline, and chloramphenicol, when appropriate, were used at final concentrations of 100 μg/ml, 50 μg/ml, 15 μg/ml, and 20 μg/ml respectively. Growth curves were performed in 96-well microtiter dishes (Becton-Dickinson, Cockeysville, Md.) using a computer-controlled Ultra Microplate Reader (Bio-Tek Instruments, Inc., Winooski, Vt.) running the KC4 software package, with incubation at 37° C. A sample of an overnight culture of S. enterica was subcultured 1:100 into minimal medium to a final volume of 200 μl supplemented with acetate at the indicated concentration. Data points were collected every 15 minutes with moderate shaking for 800 sec between readings.

Construction of plasmid pSeeutD⁺: eutD from S. enterica was amplified from the chromosome using the forward primer 5′ GTCGCCCGAATTCAACAA 3′ (SEQ ID NO:24) and the reverse primer 5′ TTAAAGGGTACCAGAACG 3′ (SEQ ID NO:25). Bases underlined indicate an EcoRI and a KpnI restriction site engineered into each primer, respectively. The resulting 1.1-kb fragment was A-tailed and gel purified using the QIAquick gel extraction kit (Qiagen, Valencia, Calif.). This product was then ligated into pGEM-T (Promega, Madison, Wis.) according to the manufacturer's instructions. The resulting plasmid contained the eutD gene in the orientation for expression from P_{lacZ}. This vector was digested with EcoRI and KpnI and the liberated 1.1-kb fragment was ligated into the EcoRI/KpnI site of pBAD30 (Guzman, et. al., J. Bacteriol. 177:4121-4130, 1995). This resulting 6.0-kb plasmid was named pSeeutD⁺.

### RESULTS

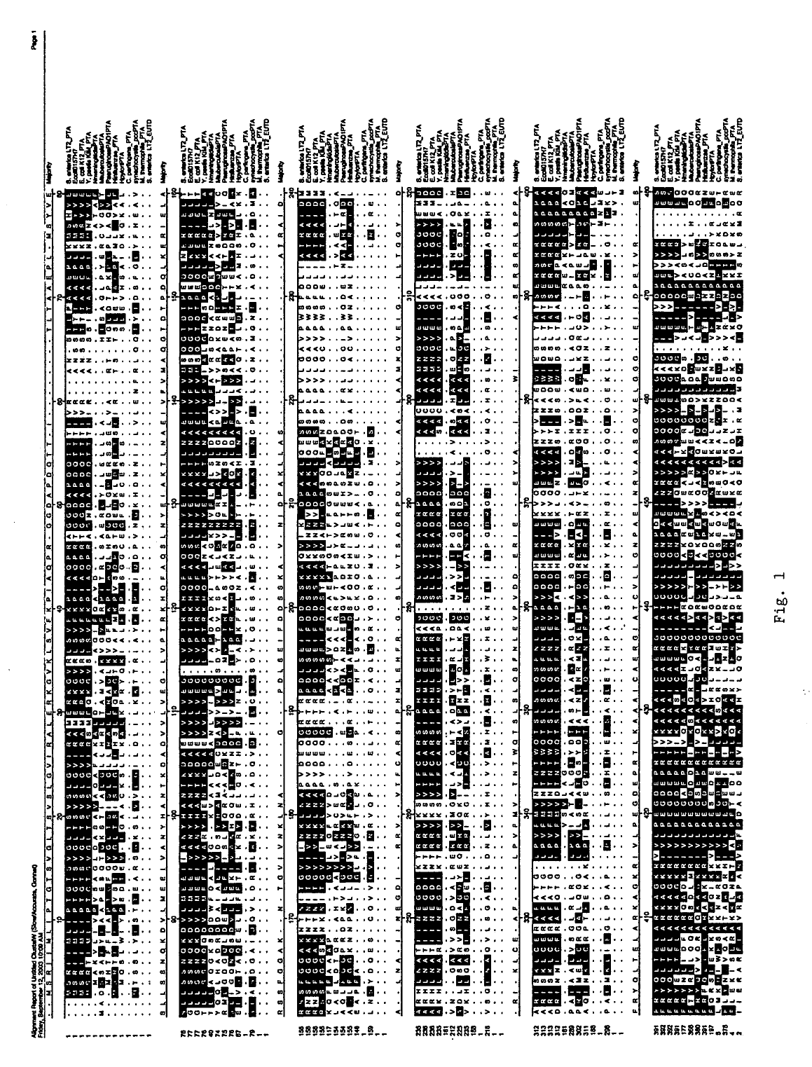

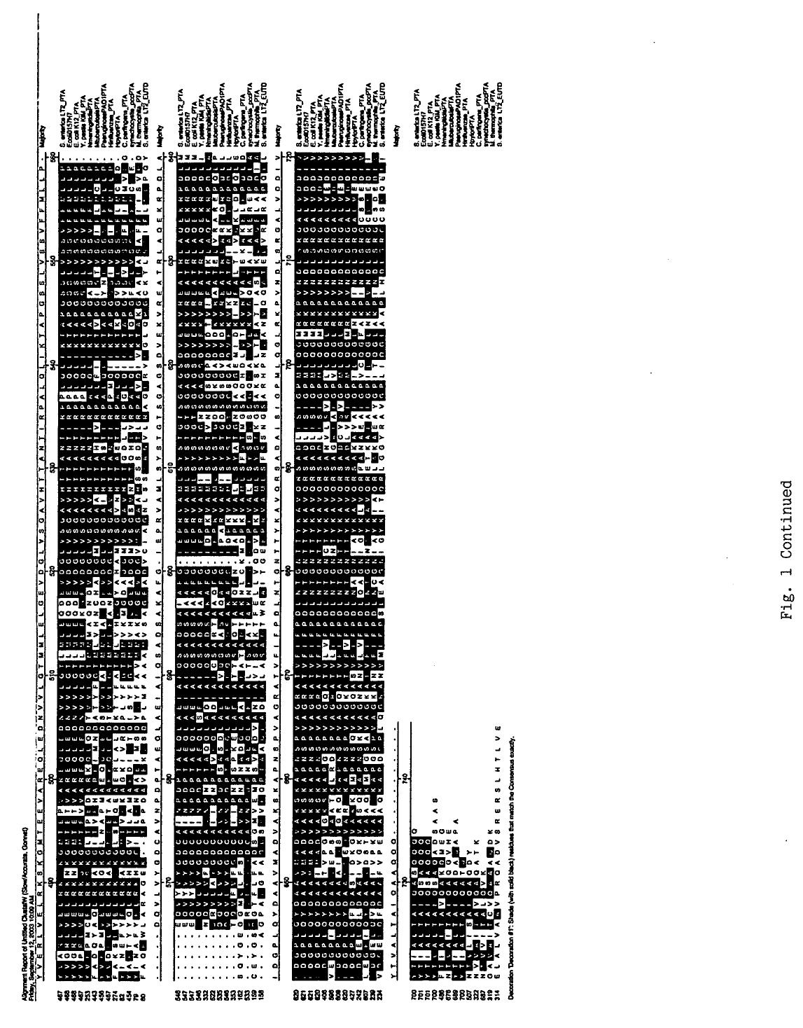

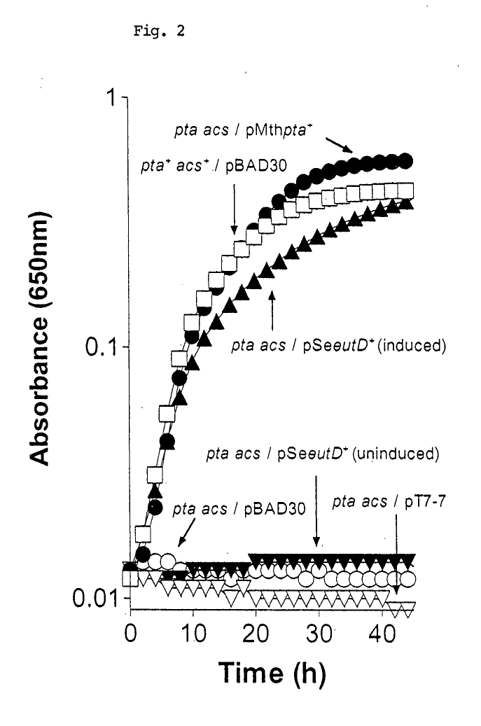

Growth curves were performed on a Salmonella enterica strain harboring lesions in both acs and pta (FIG. 2). This strain was unable to utilize acetate as a source of carbon and energy. Wildtype, as well as the double mutant transformed with a plasmid containing pta from M. thermophila grew well using 50 mM acetate as a carbon and energy source. The acetate deficient strain transformed with pSeeutD⁺ remained unable to utilize acetate under uninduced conditions, but regained the ability to utilize acetate when expression of eutD was induced in trans. Placing pMthpta in trans rescued the double mutant to optical densities better than wild type, where pSeeutD⁺ provided slightly less cell density over the same period of time.

The double mutant strain transformed with a plasmid containing pta from M. thermophila (acs/pMthpta⁺) was also tested similarly as described in example 4 to determine whether the short form Pta from M. thermophila would restore virulence. It was found that virulence was not restored. Therefore, we conclude that the N-terminal domain of a long form Pta is important for virulence.

The present invention is not intended to be limited to the foregoing examples, but encompasses all such modifications and variations as come within the scope of the appended claims.

We claim:

1. A method for identifying agents with virulence-attenuating activity on bacteria that contain a long form phosphotransacetylase, the method comprising the step of:
providing a polypeptide that comprises the N-terminal domain of the long form phosphotransacetylase;
exposing the polypeptide to a test agent; and
determining whether the agent binds to the N-terminal domain, wherein a binding indicates that the agent is likely to have virulence-attenuating activity.

2. The method of claim 1, wherein the polypeptide consists of the N-terminal domain of a long form phosphotransacetylase.

3. The method of claim 1, wherein the polypeptide is a long form phosphotransacetylase.

4. The method of claim 1, wherein all three steps are carried out in vitro.

5. The method of claim 1, where the polypeptide is provided and exposed to a test agent in a cell.

6. The method of claim 5, wherein the cell is a bacterial cell.

7. A method for attenuating virulence of a bacterium that contains the long form phosphotransacetylase, the method comprising the step of:
exposing the bacterium to a molecule that can bind to the N-terminal domain of the phosphotransacetylase at a dose sufficient to attenuate virulence.

8. The method of claim 7, wherein the bacterium is selected from P. syringae, P. aeruginosa PAO1, N. meningitidis Z2491, S. enterica LT2, E. coli 0157H7, E. coli K12, Y. pestis KIM, H. influenzae, H. pylori, Synechocystis PCC, or M. tuberculosis.

9. The method of claim 7, wherein the N-terminal domain binding molecule is an antibody to the N-terminal domain.
